(19) United States
(12) Patent Application Publication
(10) Pub. No.: US 2025/0111508 A1
(43) Pub. Date: Apr. 3, 2025

(54) Systems, Methods and Apparatuses for Computer-Aided Diagnosis of Pulmonary Embolism

(71) Applicant: Arizona Board of Regents on behalf of Arizona State University, Scottsdale, AZ (US)

(72) Inventors: Nahid UI ISLAM, Mesa, AZ (US); Zongwei ZHOU, Tempe, AZ (US); Shiv GEHLOT, Darwara (IN); Jianming LIANG, Scottsdale, AZ (US)

(21) Appl. No.: 18/904,995

(22) Filed: Oct. 2, 2024

Related U.S. Application Data
(60) Provisional application No. 63/542,056, filed on Oct. 2, 2023.

(51) Int. Cl. G06T 7/00; A61B 6/00; A61B 6/50; G06V 10/25; G06V 10/764; G06V 10/82; G06V 20/70; G16H 50/20

(52) U.S. Cl. CPC G06T 7/0012; A61B 6/504; A61B 6/5217; G06V 10/25; G06V 10/764; G06V 10/82; G06V 20/70; G16H 50/20; G06T 2200/04; G06T 2207/10081; G06T 2207/20081; G06T 2207/20084; G06T 2207/30061; G06T 2207/30101; G06V 2201/03

(57) ABSTRACT

A system comprising a memory to store instructions and a processor to execute the instructions stored in the memory to receive a plurality of Computed Tomography Pulmonary Angiography (CTPA) exams as input image data, each exam in the plurality of exams comprising a varying plurality of individual images. The system annotates each of the slices with one of a pulmonary embolism (PE) present label or PE absent label, annotates each of the exams with one of a plurality of labels each indicating a different PE state and location, performs a slice-level PE classification to determine the presence or absence of PE for each of the slices, and performs and outputs, via an Embedding-based Vision Transformer (E-ViT), an exam-level diagnosis using the slice-level classifications.

### CLAIM OF PRIORITY

This application claims the benefit of U.S. Provisional Patent Application No. 63/542,056, filed Oct. 2, 2023, entitled “SEEKING AN OPTIMAL APPROACH FOR COMPUTER-AIDED DIAGNOSIS OF PULMONARY EMBOLISM”, the disclosure of which is incorporated by reference herein in its entirety.

### GOVERNMENT RIGHTS AND GOVERNMENT AGENCY SUPPORT NOTICE

This invention was made with government support under R01 HL128785 awarded by the National Institutes of Health. The government has certain rights in the invention.

### COPYRIGHT NOTICE

A portion of this document contains material which is subject to copyright protection. The copyright owner has no objection to the facsimile reproduction by anyone of the document as it appears in the Patent and Trademark Office patent records, but otherwise reserves all copyright rights whatsoever.

### TECHNICAL FIELD

Embodiments of the invention relate generally to the field of computer-aided diagnosis of pulmonary embolism.

### BACKGROUND

Pulmonary Embolism (PE) represents a thrombus (“blood clot”), usually originating from a lower extremity vein, that travels to the blood vessels in the lungs, causing vascular obstruction, and in some patients, death. This disorder is commonly diagnosed using Computed Tomography Pulmonary Angiography (CTPA). Deep learning holds promise for Computer-aided Diagnosis (CAD) of PE. However, numerous deep learning methods, such as Convolutional Neural Networks (CNN) and Transformer-based models, exist for a given task, causing great confusion regarding the development of CAD systems for PE.

### BRIEF DESCRIPTION OF THE DRAWINGS

Embodiments are illustrated by way of example, and not by way of limitation, and can be more fully understood with reference to the following detailed description when considered in connection with the figures in which:

FIG. 1 depicts pre-processing steps for slice-level classification of an original CT scan followed by windowing and lung localization, according to embodiments of the invention;

FIG. 2 illustrates a standard image representation and vessel-oriented image representation (VOIR) in 2D, 2.5D, and 3D forms, according to embodiments of the invention;

FIG. 3 illustrates embedding-based Vision Transformer (E-ViT) for exam-level diagnosis, according to embodiments of the invention;

FIG. 4 is a graphical illustration comparing twelve architectures, in which transfer learning outperforms random initialization for slice-level PE classification, according to embodiments of the invention;

FIG. 5 illustrates a positive correlation between results on the ImageNet and RSNA PE dataset (R=0.5914), suggesting transfer learning performance can be inferred by ImageNet pre-training performance;

FIG. 6 is a SeXception attention map that highlights the potential PE location in a slice using GradCam++, according to embodiments of the invention;

FIGS. 7A and 7B illustrate evaluation of a SeXception model trained on the RSNA PE dataset on the CAD-PE Challenge Dataset and Ferdowsi University of Mashhad's PE (FUMPE) dataset;

FIG. 8 graphically illustrates that self-supervised pre-training extracts more transferable features compared with supervised pre-training, according to embodiments of the invention;

FIG. 9 graphically illustrates a performance gain with the help of SE block, according to embodiments of the invention;

FIG. 10 presents Table 2 demonstrating, in a pre-trained SeXception model, trained on the RSNA PE dataset, superior results for PE slice-level classification on two additional, unseen datasets: the CAD-PE Challenge and FUMPE, according to embodiments of the invention;

FIG. 11 presents Table 3 showing, for slice-level PE classification task, ViT models exhibit inferior performance when compared to CNNs, and conversely, Swin Transformers demonstrate performance on par with SeXception;

FIG. 12 presents Table 4 showing results of embeddings extracted by a slice-level classification model when passed through BiGRU for exam-level diagnosis;

FIG. 13 presents Table 5, in which extracted embeddings from a slice-level classification were used as input for E-ViT for the exam-level diagnosis, according to embodiments of the invention;

FIG. 14 presents Table 6 showing results of an evaluation of the E-ViT framework, including the impact of position embedding on the exam-level diagnosis, according to embodiments of the invention;

FIG. 15 presents Table 7 showing the E-ViT framework outperforms MIL-VT significantly, according to embodiments of the invention;

FIG. 16 presents Table 8 showing an evaluation of vessel-oriented image representation (VOIR) in comparison with 2D, 2.5D, and 3D solutions for the task of reducing PE false positives;

FIG. 17 presents Table 9 showing a comparison of the performance of recent 3D self-supervised methods on the 3D VOIR dataset, focusing on the task of reducing false positives;

FIG. 18 presents Table 10 showing tabular results of FIG. 4;

FIG. 19 presents Table 11 showing tabular results of FIG. 8; and

FIG. 20 presents Table 12 showing an overview of the parameter size and training speed for all models used in researching embodiments of the invention.

### DETAILED DESCRIPTION

Pulmonary Embolism (PE) represents a thrombus (“blood clot”), usually originating from a lower extremity vein, that travels to the blood vessels in the lungs, causing vascular obstruction, and in some patients, death. This disorder is commonly diagnosed using Computed Tomography Pulmonary Angiography (CTPA). Deep learning holds promise for the Computer-Aided Diagnosis (CAD) of PE. However, numerous deep learning methods, such as Convolutional Neural Networks (CNN) and Transformer-based models, exist for a given task, causing great confusion regarding the development of CAD systems for PE. To address this confusion, a comprehensive analysis of competing deep learning methods applicable to PE diagnosis based on four datasets is presented herein. First, the RSNA PE dataset is used, which includes (weak) slice-level and exam-level labels, for PE classification and diagnosis, respectively. At the slice level, CNNs are compared with the Vision Transformer (ViT) and the Swin Transformer. The impact of self-supervised versus (fully) supervised ImageNet pre-training, and transfer learning over training models from scratch, is also investigated. Additionally, at the exam level, sequence model learning is compared with a proposed transformer-based architecture, Embedding-based ViT (E-ViT), according to embodiments of the invention. For the second and third datasets, the CAD-PE Challenge Dataset and Ferdowsi University of Mashad's PE (FUMPE) Dataset are used, where (strong) clot-level masks are converted into slice-level annotations to evaluate the optimal CNN model for slice-level PE classification. Finally, an in-house PE-CAD dataset (distinct from the CAD-PE Challenge Dataset) is used, which contains (strong) clot-level masks. Here, the impact of vessel-oriented image representations and self-supervised pre-training on PE false positive reduction at the clot level across image dimensions (2D, 2.5D, and 3D) is investigated. Experiments show that (1) transfer learning boosts performance despite differences between photographic images and CTPA scans; (2) self-supervised pre-training can surpass (fully) supervised pre-training; (3) transformer-based models demonstrate comparable performance but slower convergence compared with CNNs for slice-level PE classification; (4) model trained on the RSNA PE dataset demonstrates promising performance when tested on unseen datasets for slice-level PE classification; (5) the E-ViT framework according to embodiments of the invention excels in handling variable numbers of slices and outperforms sequence model learning for exam-level diagnosis; and (6) vessel-oriented image representation and self-supervised pre-training both enhance performance for PE false positive reduction across image dimensions. An optimal approach surpasses state-of-the-art results on the RSNA PE dataset, enhancing AUC by 0.62% (slice-level) and 2.22% (exam-level). On an in-house PE-CAD dataset, 3D vessel-oriented images improve performance from 80.07% to 91.35%, a remarkable 11% gain. Code is available at GitHub.com/JLiangLab/CAD PE.

### 1. Introduction

Pulmonary embolism (PE) represents a thrombus (occasionally colloquially, and incorrectly, referred to as a “blood clot”), usually originating from a lower extremity or pelvic vein, that travels to blood vessels in the lungs and causes vascular obstruction. PE causes more deaths than lung cancer, breast cancer, and colon cancer combined (U.S. Department of Health and Human Services Food and Drug Administration, 2008).

The current test of choice for PE diagnosis is computed tomography pulmonary angiography (CTPA) (Stein et al., 2006), but studies have shown a rate of under-diagnosis at 14% and over-diagnosis at 10% with CTPA (Lucassen et al., 2013). Computer-aided diagnosis (CAD) has shown great potential for improving the imaging diagnosis of PE (Masutani et al., 2002; Liang and Bi, 2007; Zhou et al., 2009; Tajbakhsh et al., 2015; Rajan et al., 2020; Huang et al., 2020a; Zhou et al., 2019; Zhou, 2021; Zhou et al., 2021a, 2017a). However, recent research in deep learning across academia and industry has resulted in numerous architectures, various model initializations, distinct learning paradigms, and data pre-processing producing many competing approaches to CAD implementation in medical imaging, resulting in great confusion in the CAD community.

To address this confusion and develop an optimal approach, the inventors sought to answer the critical question: what deep learning architectures, model initializations, learning paradigms, and data pre-processing should be used for computer-aided diagnosis of pulmonary embolism? To answer this question, the inventors conducted extensive experiments with various deep learning methods applicable for PE diagnosis at both the slice-level and the exam-level, using a publicly available PE dataset (Colak et al., 2021a) and an in-house PE-CAD dataset (Tajbakhsh et al., 2015).

Architectures. Convolutional neural networks (CNNs) have been the default architectural choice for computer-aided diagnosis (CAD) in medical imaging (Litjens et al., 2017; Deng et al., 2020). Alternatively, transformers have proven powerful for Natural Language Processing (NLP) (Devlin et al., 2018a; Brown et al., 2020), and have been quickly adopted for image analysis (Dosovitskiy et al., 2020; Han et al., 2021; Touvron et al., 2021), leading to Vision Transformer (ViT) (Dosovitskiy et al., 2020; Vaswani et al., 2017). ViT has shown competitive performance for medical imaging applications such as classification (Xiao et al., 2023) and segmentation (Shamshad et al., 2022). The discussion herein assesses the performance of the original ViT, Swin Transformer and 12 CNN variants for the slice-level PE classification task. The ensemble of Xception, SeXception, and SeResNext50 architecture (CNN-based architectures) demonstrates an improvement in slice-level PE classification performance, achieving gains of 4.97% and 0.30%, respectively, over the performance of the original transformer-based models.

Model initializations. Training deep models generally requires massive carefully annotated training datasets (Tajbakhsh et al., 2021; Haghighi et al., 2021). However, it is often prohibitive to create such large annotated datasets in medical imaging. Due to the lack of a sufficiently large annotated dataset, training a model from scratch may lead to suboptimal performance. Transfer learning provides a data-efficient alternative to this problem, whereby a model pre-trained on a source task (e.g., ImageNet) is fine-tuned on the related, but different, target task. Pre-training and fine-tuning have proven more effective than training models from scratch (Tajbakhsh et al., 2016; Zhou et al., 2017b). There are two major strategies to pre-train models (fully-supervised and self-supervised learning). Fully-supervised learning pre-trains a model using annotated data, whereas self-supervised learning does not require annotation (Jing and Tian, 2020; Haghighi et al., 2020). As disclosed herein, the inventors benchmark 12 different CNN architectures with fully-supervised pre-trained weights and evaluate 19 self-supervised methods for slice-level PE classification. Both SeLa-v2 (Asano et al., 2019a) and DeepCluster-v2 (Caron et al., 2018) self-supervised methods surpass their fully-supervised counterparts with ˜0.95% gain. The inventors also evaluate Models Genesis (Zhou et al., 2021b) for the task of reducing false positives in 3D volume-based PE detection, achieving a performance improvement of 1.13% compared to training from scratch.

Learning paradigms. For exam-level PE diagnosis, predictions are made for a collection of slices of CT scans. Due to the acquisition mechanism, a spatial correlation among the slices exists for each exam. Sequence model learning such as the recurrent neural network, long-and short-term memory, and the gated recurrent unit can exploit this spatial correlation in the datasets, leading to improved exam-level diagnosis performance. Bidirectional Gated Recurrent Unit was attempted for the task of exam-level PE diagnosis. The application of a transformer-based model for exam-level diagnosis was also investigated. Instead of following the traditional methods, the ViT architecture was adopted to handle slice-level embeddings, which offers a more efficient and flexible solution compared to raw images for exam-level diagnosis. This approach overcomes the limitations of high-dimensional images and enables the Trans-former encoder to process an arbitrary number of slices for each patient. Similar to MIL-VT (Yu et al., 2021), embodiments utilize both class embedding and exam-level embedding for exam-level diagnosis. This approach achieves an 1.53% gain over the previous state-of-the-art performance for exam-level PE diagnosis.

Data pre-processing. Representation of input data is critical for machine learning algorithms, and an optimal representation may significantly enhance performance. To this end, vessel-oriented image representation (VOIR) (Tajbakhsh et al., 2015) is explored compared with standard image representation for the task of reducing PE false positives. As an overview, VOIR follows these steps: 1) the generation of candidates, 2) vessel axis determination, and 3) selection of slices. In effect, VOIR can be used as a pre-processing step to obtain the performance gain over the conventional representation. Embodiments extend VOIR (Tajbakhsh et al., 2019) into its 3D form as a data pre-processing. Experiments were conducted to demonstrate its effectiveness for the task of reducing PE false positives. VOIR outperforms standard image representation in 3D volume-based data with an AUC gain of 11.28%.

### 2. Related Work

Earlier work in automated PE diagnosis focused on using pulmonary ventilation scans; the scans were passed through a neural network for PE detection (Patil et al., 1993; Tourassi et al., 1995; Scott and Palmer, 1993; Serpen et al., 2008), achieving modest success, but, with inadequate generalizability. Subsequently, CTPA scans replaced ventilation-perfusion scans for PE detection (Liang and Bi, 2007; Özkan et al., 2014; Park et al., 2010). However, these approaches were based on manual feature engineering, making them suboptimal and computationally complex. Recent advancements in deep learning led to end-to-end deep learning models for PE diagnosis, e.g., CNNs were used by Tajbakhsh et al. (2015). Additionally, the input is a vessel-aligned image representation rather than a standard representation. As opposed to 2D CNNs, Yang et al. (2019) divided the CTPA scan into smaller cubes and evaluated the cubes using 3D CNNs. Huang et al. (2020b) proposed PENet, a 3D CNN that uses multiple slices for PE prediction. Furthermore, Shi et al. (2020) utilized a two-stage PE detection framework that outperformed the PENet (Huang et al., 2020b). In the first stage, a CNN was trained with attention supervision using small pixel-level annotated slices, and in the second stage, an RNN outputted patient-level PE prediction. Rajan et al. (2019) also explored a two-stage framework, in which the first stage generated the PE candidates, and the second stage utilized multiple instance learning (MIL) for PE detection. Similarly, Suman et al. (2021) used a two-stage framework to provide slice-and exam-level diagnosis, utilizing MIL in the second stage.

The top performing methods in the Radiological Society of North America (RSNA) Pulmonary Embolism Detection Challenge (Colak et al., 2021b) also utilized a two-stage framework for slice-level PE classification and exam-level diagnosis. For example, the first-place solution used a CNN in the first stage for slice-level classification. Subsequently, the slice-level embeddings from the CNN model were passed through a bidirectional gated recurrent unit to output exam-level diagnosis. Similarly, the second-place solution used two CNNs for slice-level embedding extraction, which were fed to DistilBERT (Sanh et al., 2019) for exam-level diagnosis. However, this approach was computationally expensive due to the incorporation of multiple CNNs. The third-place solution was similar to the first-place solution, albeit with two significant differences: 1) a penultimate layer was used for predicting seven fine-grained PE labels in the first stage, and 2) bidirectional LSTM replaced bidirectional GRU in the second stage.

Islam et al. (2021) first presented a benchmark on slice-level classification in the first stage and exam-level diagnosis in the second stage using the RSNA PE dataset (Colak et al., 2021b). Embodiments significantly extend the preliminary version substantially with the following enhancements:
1. An extensive benchmark with 12 fully-supervised and 19 self-supervised pre-trained models for slice-level PE classification (see § 4.1).
2. A novel Embedding-based Vision Transformer (E-ViT) for exam-level PE diagnosis, according to the disclosed embodiments. Unlike the original ViT, the encoder in E-ViT exploits both class embedding and exam-level embedding, resulting in a significant exam-level performance gain (see § 4.2.2).
3. A comprehensive comparison between sequence model learning and E-ViT for the exam-level PE diagnosis (see § 5.2).
4. A comparative analysis of the impact of vessel-oriented image representations and model initialization on PE false positive reduction in 2D, 2.5D, and 3D (see § 3.4).

### 3. Materials

### 3.1. RSNA PE Data

This dataset consists of 7,279 CTPA exams, with a varying number of individual images or slices in each exam, using an image size of 512×512 pixels. The test set is created by randomly sampling 1,000 exams, and the remaining 6,279 exams form the training set. Correspondingly, there are 1,542,144 and 248,480 slices in the training and test sets, respectively. This dataset is annotated at both slice and exam-levels; that is, each slice has been annotated as either PE present or PE absent, and each exam has been further annotated with an additional nine labels, i.e., negative exam for PE, indeterminate, left PE, right PE, central PE, right ventricular/left ventricular (RV/LV) ratio >1, RV LV ratio <1, chronic PE and acute, and chronic PE.

Pre-processing. Similar to the first-place solution for the RSNA Pulmonary Embolism Detection Challenge (Colak et al., 2021b), lung localization and windowing have been used as pre-processing steps for slice-level classification. Lung localization removes irrelevant tissues and keeps the region of interest in the slices, whereas windowing highlights the pixel intensities within the range of [100-700 HU (Hounsfield Units)]. Also, the slices are resized to 576×576 pixels. FIG. 1 illustrates these pre-processing steps in detail, where the original CT scan is depicted at 100, the CT scan after windowing is depicted at 105, and the CT scan after lung localization is depicted at 110. Three adjacent slices from an exam are considered as the 3-channel input of the model.

### 3.2. CAD-PE Challenge Dataset

The CAD-PE Challenge Dataset comprises 91 computed tomography pulmonary angiograms (CTPA) that have been positively diagnosed with pulmonary embolism (PE) by at least one experienced radiologist (González et al., 2020). There are altogether 41,256 slices. Each scan has been segmented to highlight all the clots. The dataset was created for the IEEE International Symposium on Biomedical Imaging (ISBI) challenge.

Pre-processing. Embodiments take advantage of the provided ground truth masks to generate slice-level annotations. Similarly, embodiments apply the same pre-processing steps to the dataset as for the RSNA PE dataset. Embodiments leverage the best CNN model trained on the RSNA PE dataset to assess the model's performance using the CAD-PE challenge dataset (§ 5.1.1).

### 3.3. Ferdowsi University of Mashhad's PE (FUMPE) Dataset

FUMPE is a publicly available dataset that includes three-dimensional CTPA images of 35 patients, with a total of 8,792 slices. For each image, two expert radiologists provided segmentation ground truths to identify the PE regions (Masoudi et al., 2018).

Pre-processing. The FUMPE dataset includes segmentation masks that highlight the regions of PE. The dataset is investigated to identify which slices have mask values, and consider slices with mask values to be PE positive. Embodiments apply the same pre-processing procedure to the FUMPE dataset as for the RSNA PE dataset. The optimal CNN model trained on the RSNA PE dataset is used to evaluate the model's performance on the FUMPE dataset (§ 5.1.1).

### 3.4. PE-CAD Dataset

An in-house PE-CAD dataset (not to be confused with the CAD-PE Challenge dataset described herein) was annotated at the clot-level, that is, each clot was manually segmented. The dataset is comprised of 121 CTPA scans with 326 emboli and the spatial coordinate information of these emboli. At the patient level, the dataset is separated into a training set (71 patients) and a test set (50 patients). This dataset is analyzed in two stages for PE detection: PE candidate generation and false positive reduction.

These two stages have been extensively covered in the existing literature. For example, Models Genesis (Zhou et al., 2019), a self-supervised model, was fine-tuned for a PE-CAD dataset as a target dataset. Here, Models Genesis was pre-trained on a dataset of chest CT scans called LUNA16 (Setio et al., 2016), achieving an AUC of 87.20±2.9 for the task of PE false positive reduction. On the other hand, Parts2Whole (Feng et al., 2020) and TransVW (Haghighi et al., 2021) also evaluated their self-supervised methods on the PE-CAD dataset achieving AUCs of 86.14±2.9 and 87.07±2.8 via fine-tuning pre-trained models, respectively. The disclosed embodiments, in addition to standard image representations, also utilize the vessel-oriented image representation of this dataset as described in Tajbakhsh et al. (2019) and extend their image representation to full 3D (see pre-processing) for PE false positive reduction. To conduct a fair comparison with these prior studies, candidate-level AUC is computed by classifying true positives and false positives.

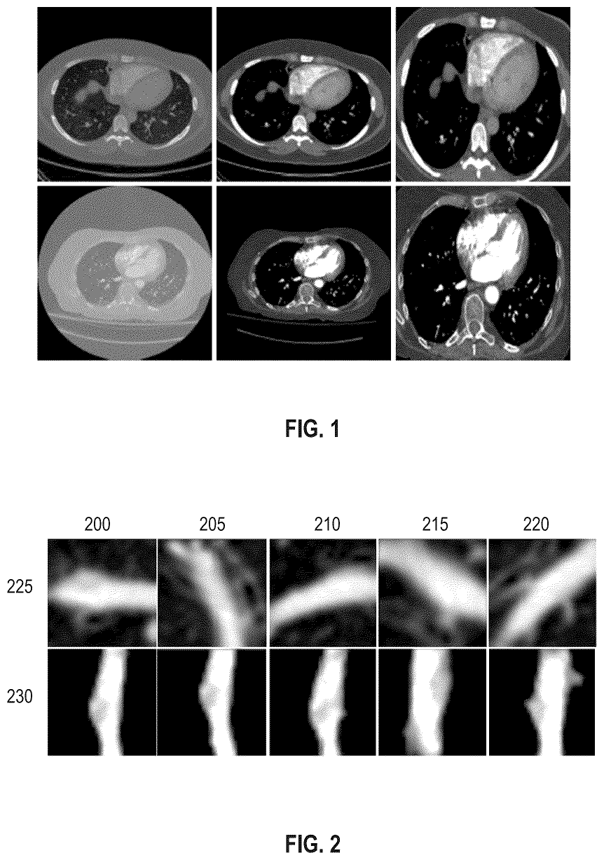

Pre-processing. The approach developed in Liang and Bi (2007); Bi and Liang (2007) has been utilized for generating PE candidates based on heuristic lung segmentation and the tobogganing algorithm (Fairfield, 1990). The lung appears hypoattenuating or darker than its surroundings in chest CTPA scans. Therefore, to separate the lungs from the remainder of the scan, the voxel intensity values are clipped using a −400 HU threshold, resulting in a binary volume with the lungs and other dark regions appearing hyperattenuating or white. Then, a closing operation is performed to fill all dark gaps in the white region. A 3D connected component analysis eliminates non-lung areas, removing components with small volumes or a considerable length ratio between the major and minor axes. The segmentation of the lungs is intended to reduce computational time and the frequency of false positives for the tobogganing algorithm. As PEs exclusively appear within pulmonary arteries, exploring PE candidates outside the lungs is not required. The tobogganing algorithm is then applied specifically to the lung area, generating the PE candidate coordinates used to crop sub-volumes from the CTPA scan. The candidate generation process is detailed in (Liang and Bi, 2007; Bi and Liang, 2007). After candidate generation, each candidate is labeled as “PE” or “non-PE” based on the clot-based ground truth masks. Tajbakhsh et al. (2015) developed a 3D vessel-oriented image presentation: A principal component analysis was performed to determine the vessel axis and the two orthogonal directions. By rotating the orthogonal direction, a number of cross-sectional and longitudinal image planes were obtained. Finally, a 3-channel image representation was created for each PE candidate by selecting the middle slices from each axis. This 3-channel image representation of each PE candidate is used to form the 2.5D orthogonal PE data. The 2D slice-based PE data was formed by copying the middle slice of the z-axis 3 times. Last, each of the candidates themselves from the patients was used as 3D volume-based PE data. FIG. 2 shows both standard PE representation (row 225) and vessel-oriented PE representation (row 230) appearances for PEs 200, 205, 210, 215 and 220. It should be noted that the 2.5D image presentation is equivalent to that reported by Tajbakhsh et al. (2015), while the 2D image presentation is included in this paper as a baseline, and the 3D vessel-oriented image presentation has not been used in earlier studies.

### 4. Methods

### 4.1. Slice-Level Classification in RSNA PE Dataset

Slice-level classification refers to determining the presence or absence of PE for each slice. This section describes the configurations of fully-supervised and self-supervised transfer learning in the disclosed embodiments.

### 4.1.1. Fine-Tuning Fully-Supervised Pre-Trained Models

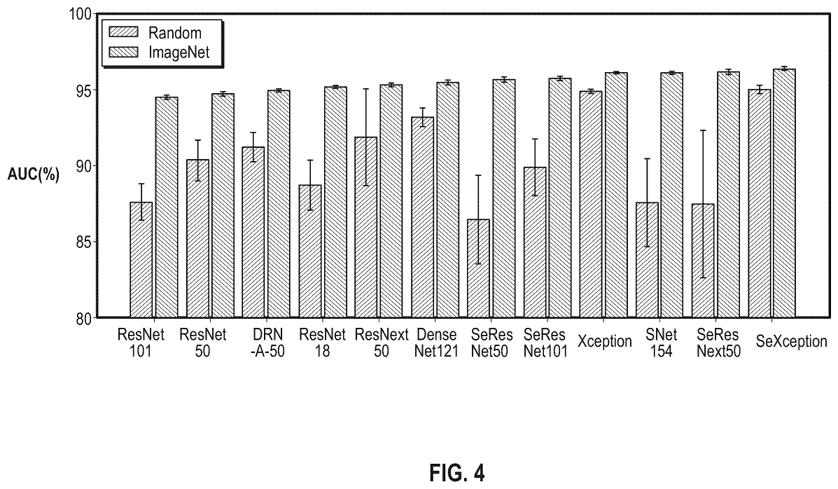

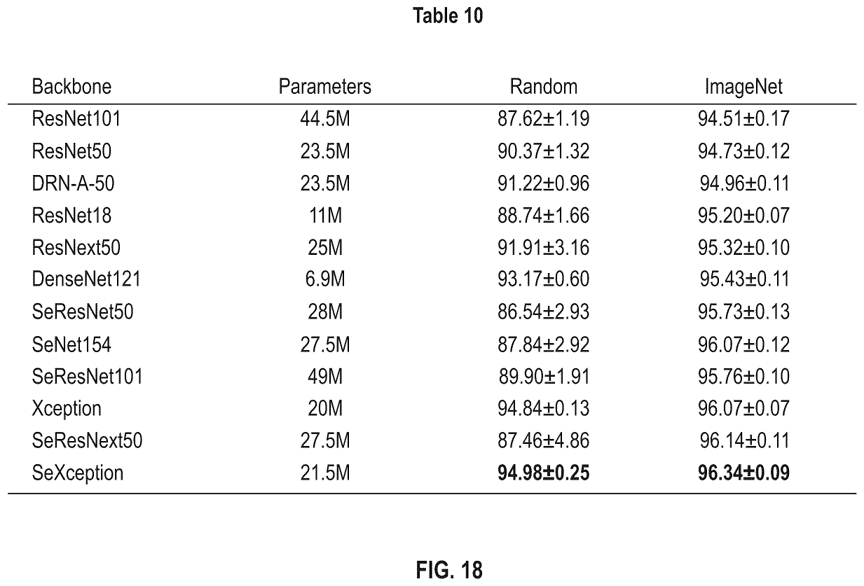

In transfer learning, a model pre-trained on a different source task is fine-tuned on the target dataset. In this set of experiments, models pre-trained on ImageNet under a fully-supervised setting are fine-tuned for PE slice-level classification using the RSNA PE dataset. For this purpose, 12 different CNN architectures (FIG. 4) are examined: Vision Transformer (ViT), and Swin Transformer. With reference to FIG. 4, for all 12 architectures, transfer learning outperforms random initialization for slice-level PE classification, despite the pronounced difference between ImageNet and RSNA PE datasets. Mean AUC and standard deviation over 10 runs are reported for each architecture. Compared with the previous state of the art (SeResNext50), the SeXception architecture achieves a significant improvement (p=1.68E-4). Also, inspired by SeResNext50 and SeResNet50, embodiments utilize squeeze and excitation (SE) blocks to the Xception architecture (SeXception, which is trained on ImageNet from scratch and other models are from PyTorch). FIG. 18 displays table 10 which provides the tabular results of FIG. 4.

Embodiments use only one epoch to train the models. The learning rate, batch size, and optimizer is 0.0004, 20, and Adam, respectively. Four V100 GPUs were used to train and test the models. The inventors also explored the usefulness of ViT, in which the slices are reshaped into a sequence of patches. Upscaling the slice for a given patch size will effectively increase the number of patches, thereby enlarging the size of the training dataset. Similarly, the number of patches increases with a decrease in the patch size. Hence, to explore these two characteristics, the inventors experimented with 32×32 and 16×16 patches, respectively (Devlin et al., 2018b), and also with slices of different sizes. Here, the inventors explored both Vision Transformer (ViT) and Swin Trans-former pre-trained on ImageNet1k and ImageNet21k.

### 4.1.2. Fine-Tuning Self-Supervised Pre-Trained Models

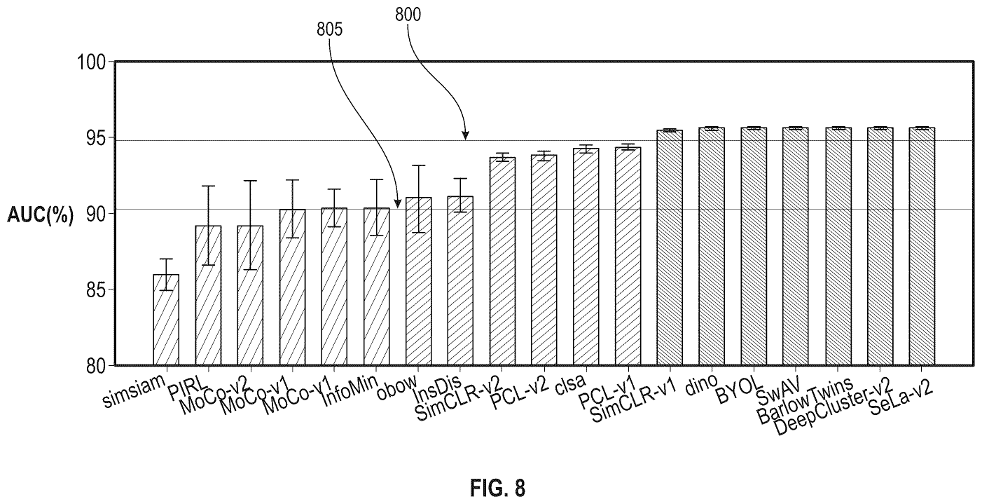

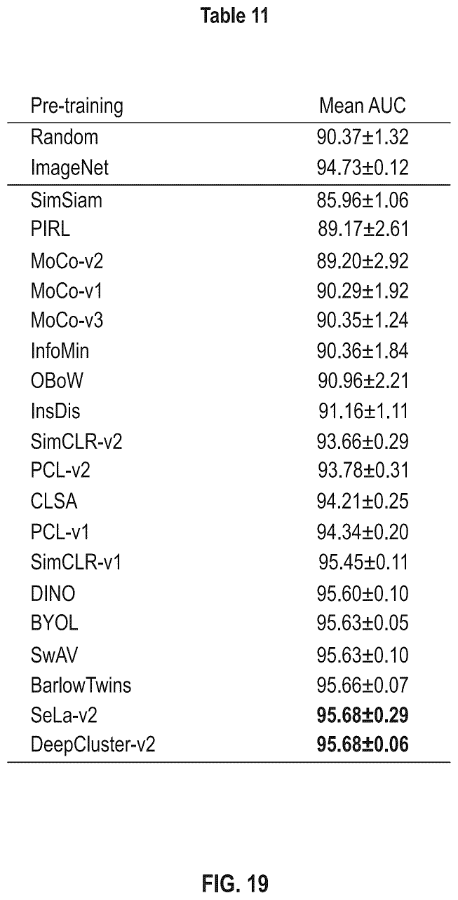

Self-supervised learning eliminates the need for explicit annotations by training the model on a pretext task. The pre-trained model can then be fine-tuned on downstream tasks such as classification and segmentation, as demonstrated in (Hu et al., 2021). One example of a pretext task is reconstructing the original image from a distorted version using strong augmentations. When it comes to transfer learning, pre-trained SSL models can be used to initialize the model's weights, instead of using randomly initialized weights or weights from supervised ImageNet models. A study collected 19 publicly available pre-trained SSL models and fine-tuned them on the RSNA PE dataset for the task of PE slice-level classification as shown in FIG. 8, where self-supervised pre-training extracts more transferable features compared with supervised pre-training. Line 800 represents the AUC of supervised pre-training, while line 805 represents the AUC of learning from scratch, with the shaded area above and below line 805 indicating the standard deviation. Seven of 19 SSL methods (the rightmost 7 columns in the graph) outperform the supervised pre-training. Here, the figure illustrates a comparison of results obtained using ResNet50 as the backbone, as only the SSL pre-trained models are publicly available. All the models used ResNet50 as the backbone architecture. The aim of this experiment is to compare the performance of models initialized with self-supervised learning against those initialized with fully-supervised learning, in order to understand the benefits of using SSL models in transfer learning. FIG. 19 displays table 11 which provides the tabular results of FIG. 8.

### 4.2. Exam-Level Diagnosis in RSNA PE Dataset

In addition to slice-level classification, the RSNA PE dataset also provides exam-level labels, in which only one label is assigned for each exam. Embodiments used the slice-level embeddings extracted from the models trained for slice-level PE classification for the data pre-processing step. All the extracted slice-level embeddings are stacked together, resulting in an N x 2048 embedding for each exam, where N denotes the number of slices per exam. Following the first-place solution, the embedding difference between the current and the two direct neighbors is computed and concatenated with the current embedding of the slice. Therefore, the input for the exam-level diagnosis task is expanded to 6144 (2048×3). On the other hand, the number of slices per exam varies from exam to exam. To address this variability, the slice-level embeddings are reshaped to K×6144 through padding or resizing. The first-place solution fixed K to be 192. One approach is to take advantage of the varying number of slices per exam, rather than standardizing it. For the exam-level diagnosis task, two learning paradigms were explored in the following subsections.

### 4.2.1. Sequence Model Learning

As for sequence model learning, bidirectional Gated Recurrent Unit (BiGRU) was employed. BiGRU is a sequence model consisting of two GRUs (Cho et al., 2014). One GRU takes an input sequence in a forward direction while the other one proceeds in a backward direction. The first-place solution in the RSNA challenge used a single bidirectional Gated Recurrent Unit (BiGRU) to generate an output sequence. The output sequence goes through a max pooling and an attention weighted average pooling and then is concatenated as an exam-level embedding. The exam-level embedding undergoes 10 separate classification layers to predict nine exam-level labels and one slice-level label. The hidden size of the BiGRU is 512, and the architecture is trained for 25 epochs with a batch size of 64. The current implementation of the classification layer in the BiGRU model, which predicts slice-level labels, is flawed. As the number of slices per exam is inconsistent, the design uses linear interpolation on the slice-level ground truth to achieve a fixed size of 192, but in doing so, the expert annotation is compromised. A more effective approach would be to adopt a framework that is capable of handling variable numbers of slices and incorporates expert annotation to ensure more accurate predictions.

### 4.2.2. Embedding-Based Vision Transformer (E-ViT)

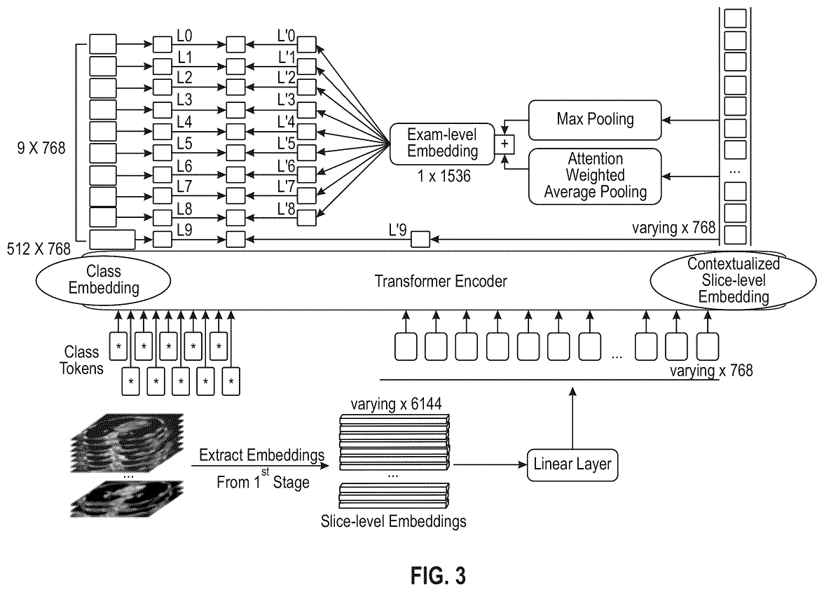

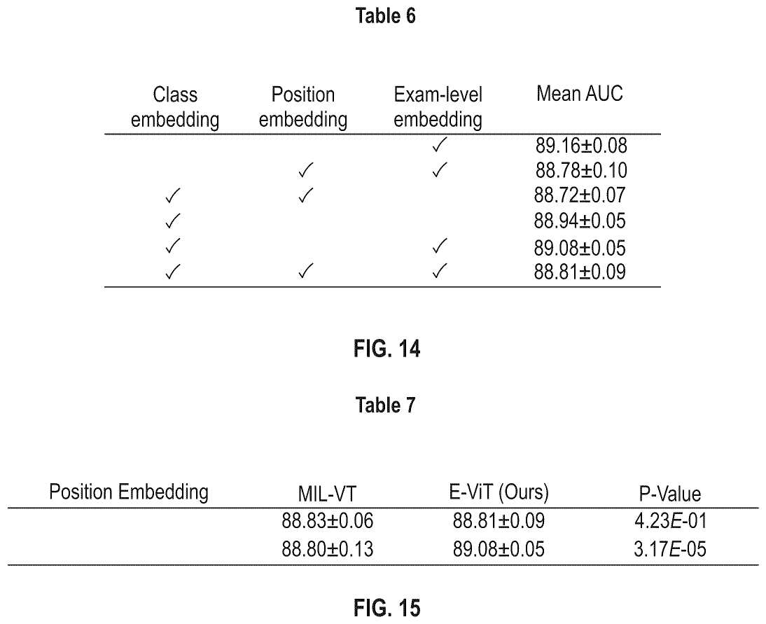

Embodiments modify Vision Transformer (ViT) (Dosovitskiy et al., 2020) for the exam-level diagnosis as illustrated in FIG. 3, referred to herein as Embedding-based Vision Transformer (E-ViT) for exam-level diagnosis. Unlike the original ViT architecture (Dosovitskiy et al., 2020), E-ViT employs a sequence of slice-level embeddings and multiple class tokens as input and discards the position embedding. Here, the slice-level embeddings are extracted from the trained models in the PE slice-level classification task (see § 4.2). Subsequently, the Transformer encoder of E-ViT outputs both class embeddings, and contextualized slice-level embeddings. The contextualized slice-level embeddings then go through attention weighted average pooling and max pooling to form an exam-level embedding via concatenation. In summary, E-ViT integrates class embedding with exam-level embedding by utilizing the varying number of slices per exam, resulting in an enhanced feature representation extracted from individual sequences. First, E-ViT takes a sequence of slice-level embeddings rather than image patches as input. The slice-level embeddings are extracted from the task of slice-level classification (§ 4.1), so they are more discriminative and informative than raw CT slices. The E-ViT framework works with a varying number of slices, unlike the first-place solution which uses a fixed number of slices. Second, 10 class tokens are used (nine for each exam label and one for slice label) to address the multi-label classification task. Having the slice label alongside exam labels enforces the consistency between exam-level and slice-level predictions. Third, E-ViT discards the position embedding for each CT slice because neighboring slices in a CT scan appear similar; therefore, retaining the position embedding can lead to exam-level performance degradation, as evidenced in Table 6 displayed in FIG. 14. In summary, E-ViT integrates class embedding with exam-level embedding to fully exploit the feature representations extracted from individual sequences with varying numbers of slices.

Embodiments apply the same pre-processing to the embeddings extracted from the slice-level classification stage (presented in § 4.2.1). Embodiments then incorporate a linear layer to reduce the slice-level embedding dimension from V×6144 to V×768 as the input of the Transformer encoder. Here, V represents the varying number of slices per exam as the number of slices is not consistent across different exams. Through investigation of the RSNA PE dataset, it was found that the upper bound of V can be set to 512, as it is sufficient for the detection of PE within the lung area. When working with varying numbers of slices per exam, it's important to consider how best to utilize the available data in order to ensure accurate predictions. In one approach, if the number of slices for a particular exam is greater than 512, the attention masking mechanism from the BERT implementation (Devlin et al., 2018a) is used to ensure that only the relevant slices are used in the model. This allows focusing on the most relevant information and improves the efficiency of the model. On the other hand, if the number of slices for a particular exam is above 512, it is assumed that most likely the slices contain regions other than the lungs. Therefore, the 512 slices belonging to the lung area are used to ensure that the model is only processing relevant information. This approach allows effective handling of varying numbers of slices and optimizes the use of the available data.

The Transformer encoder uses a multi-head self-attention mechanism to output contextualized slice-level embedding (sized V×768) and class embedding (sized 10×768). The contextualized slice-level embedding is concatenated and undergoes attention weighted average pooling and max pooling together. The concatenated exam-level embedding is then fed to 10 separate classification layers to predict the nine exam-level labels and one slice-level label. Similarly, each class embedding is utilized to predict the corresponding label through 10 different classification layers. The class embedding and exam-level embedding is integrated using the formula Avg(CE, AMP(EE)). Here, CE represents class embedding, and AMP (EE) represents the exam-level embedding (EE) which is the concatenation output of attention weighted average pooling and max pooling for contextualized slice-level embedding from the Transformer encoder. This architecture is referred to herein as the Embedding-based Vision Transformer (E-ViT). Also, this architecture is trained with the mean binary cross entropy loss from exam-level embedding and class embedding predictions. During inference, the mean of probability score from these two branches is utilized for making the final exam-level diagnosis. In the initial set of hyperparameters, the original Vision Transformer (Dosovitskiy et al., 2020), specifically ViT-B, is used. However, to further optimize the performance of E-ViT model, the inventors conducted several experiments to find the best combination of hyperparameters such as learning rate, optimizer, batch size, and total number of epochs. According to the experiments, the best combination of hyperparameters for the E-ViT model are:

TABLE 1
Hyperparameter settings used for the E-ViT model.

| Hyperparameter | Value |
|---|---|
| Learning Rate | 0.001 |
| Optimizer | Adam |
| Batch Size | 32 |
| Learning Rate Scheduler | ReduceLROnPlateau |
| Total Number of Epochs | 100 |

### 4.3. False Positive Reduction in PE-CAD Dataset

As discussed in § 3.4, the PE candidates are generated with location coordinates. The sub-volumes are cropped to ensure that PE candidates are in the center of each sub-volume. Candidate-level AUC is computed for classifying true positives and false positives for comparison with prior studies (Zhou et al., 2017a; Tajbakhsh et al., 2016, 2019). The significance of vessel-oriented representation over the standard image representation is highlighted. ResNet18 is incorporated as the backbone for PE false positive reduction, and performance with training from scratch and transfer learning with ImageNet weights is evaluated. A learning rate of 0.001, a batch size of 32, Adam as an optimizer, and patience as 38, is used. The early stopping technique is used to monitor the validation loss. The image size for 2D and 2.5D input data is 224×224, while an image size of 64×64×64 was used for 3D input data.

### 5. Results and Discussions

This section presents an analysis of the slice-level classification and exam-level diagnosis on the RSNA PE dataset, followed by the PE false positive reduction on the PE-CAD dataset.

### 5.1. Slice-Level Classification

### 5.1.1. Fine-Tuning Pre-Trained Models Outperforms Training From Scratch

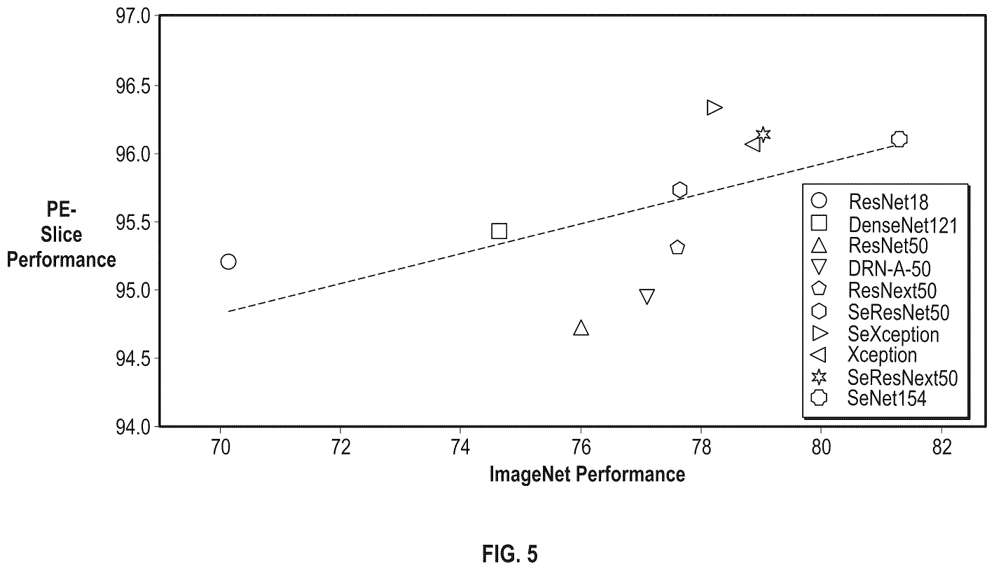

FIG. 4 shows a significant performance gain for every pre-trained model compared with random initialization. There is also a moderate positive correlation of 0.5914 between ImageNet and PE classification performance across different architectures. FIG. 5 shows there is a positive correlation between the results on ImageNet and RSNA PE dataset (R=0.5914), suggesting that the transfer learning performance can be inferred by ImageNet pre-training performance). The correlation indicates that the useful weights learned from ImageNet can be successfully transferred to the PE classification task, despite the modality difference between photographic images (ImageNet) and CTPA scans (RSNA PE). To demonstrate the interpretability of the trained model, GradCam++ (Gildenblat and contributors, 2021) is used to visualize the attention map of the SeXception architecture, which performed the best as a standalone model.

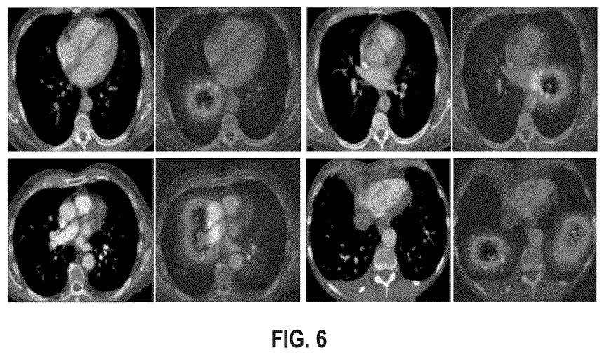

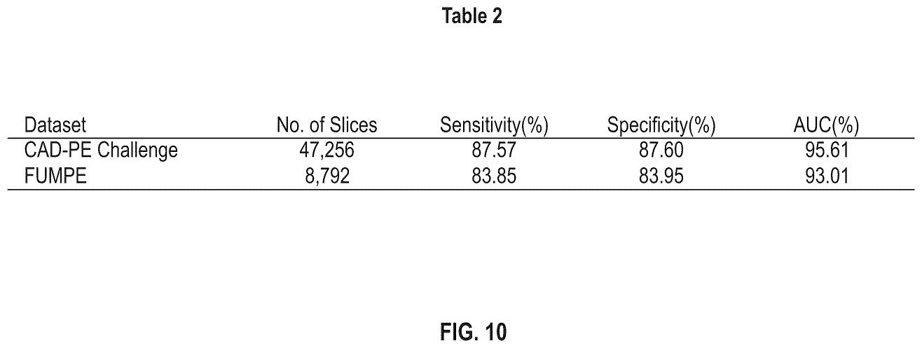

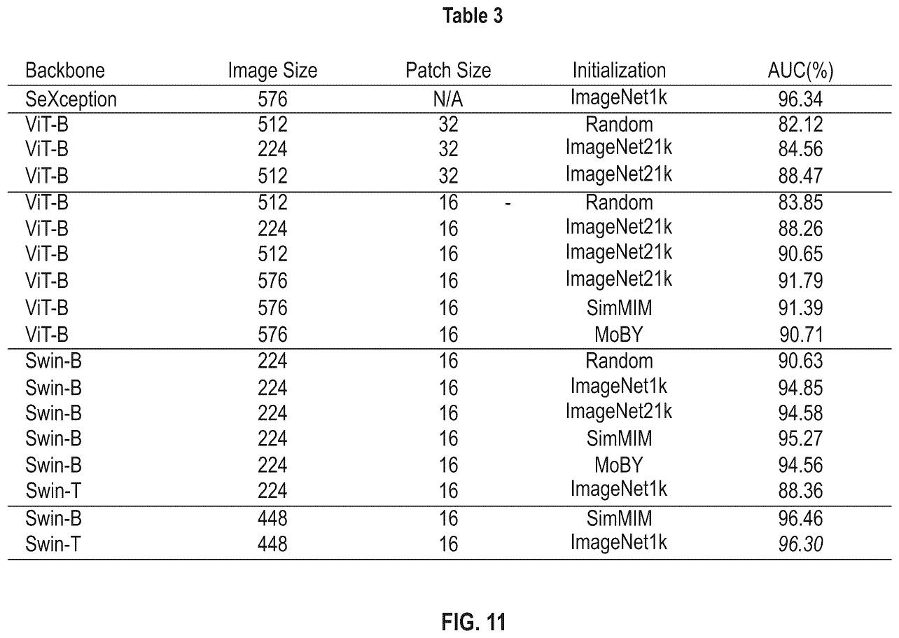

As shown in FIG. 6, the attention map effectively highlights the potential PE location in the slice. Additionally, to assess the generalizability of the SeXception model trained on the RSNA PE dataset, the model was directly tested on two additional datasets: CAD-PE (González et al., 2020) and FUMPE (Masoudi et al., 2018). The results are summarized in Table 2, displayed in FIG. 10. In Table 2, the pre-trained SeXception model, which was trained on the RSNA PE dataset, demonstrates promising results for PE slice-level classification on two additional, unseen datasets: the CAD-PE Challenge and FUMPE.

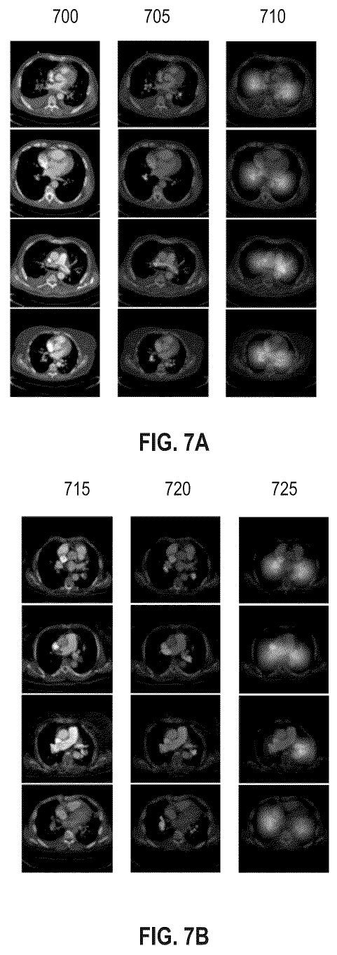

As shown in FIGS. 7A and 7B, the GradCam++ highlights the PE region 700 for the CAD-PE Challenge dataset and PE region 715 for the FUMPE dataset. The figures show the ground truth (columns 705 and 720) and predicted PE region (columns 710 and 725). FIGS. 7A and 7B show promising results for localizing the PE region on the two unseen datasets.

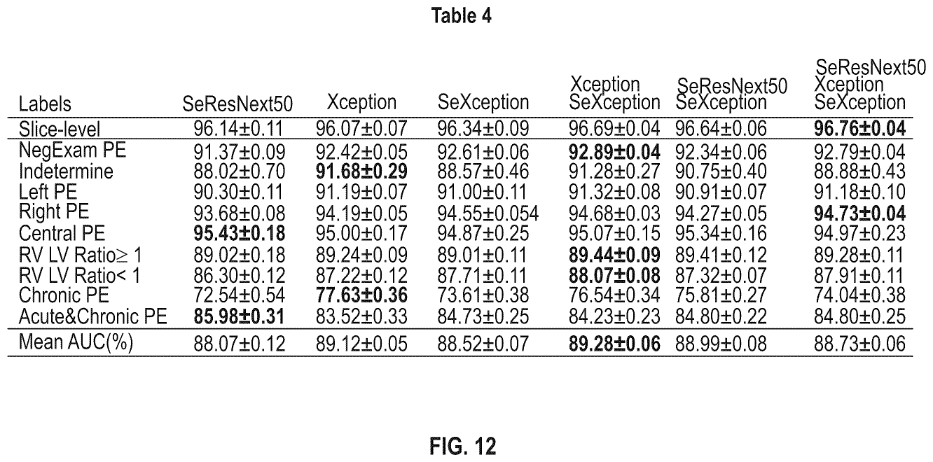

It is noteworthy that the model was only trained on the RSNA PE dataset and had not seen the two additional datasets prior to evaluation. The inventors evaluated the two additional datasets for slice-level classification to determine whether a slice has PE or not. Here, the model had not seen the segmentation ground truth for these datasets either. Despite this, the model not only accurately classified slices with PE but also efficiently localized them. The highlighted area in 705 of FIG. 7A and the highlighted area in 720 of FIG. 7B represents the ground truths of the given segmentation mask, whereas the highlighted area in 710 of FIG. 7A and the highlighted area in 725 of FIG. 7B represents the predicted PE region. The experiments demonstrate not only the feasibility of using deep learning for diagnosing challenging radiologic findings such as PE on CTPA, but also the ability to accommodate data from external institutions employing different CT scanners and imaging protocols. Moreover, the superior performance of fine-tuning pre-trained models highlights the importance of transfer learning in medical image analysis and could have implications for improving the diagnostic accuracy and efficiency of PE detection in clinical practice. The SeXception model, trained on the RSNA PE dataset, demonstrates strong performance in PE slice-level classification on the CAD-PE Challenge and FUMPE datasets. According to Table 2, the SeXception achieves a sensitivity of 87.57% and a specificity of 87.60% on the CAD-PE Challenge dataset, and a sensitivity of 83.85% and a specificity of 83.95% on the FUMPE dataset. The slice-level classification result is combined on the RSNA PE dataset by taking the average from Xception and SeXception, SeResNext50 and SeXception, and SeResNext50 and Xception and SeXception as displayed in FIG. 12, Table 4. The ensemble of SeResNext50 and Xception and SeXception achieves the best AUC performance of 96.76±0.05 gaining 0.62% compared with the first-place solution.

### 5.1.2. Self-Supervised Pre-Training can Surpass Fully-Supervised Pre-Training

As summarized in FIG. 8, SeLa-v2 (Asano et al., 2019b) and DeepCluster-v2 (Caron et al., 2018) have achieved the best AUC of 95.68±0.05 and 95.68±0.06, followed by Barlow Twins (Zbontar et al., 2021a). Seven of 19 self-supervised ImageNet models performed better than supervised pre-trained ResNet50 (FIG. 8). Six self-supervised methods underperform fully-supervised pre-trained ResNet50 but outperform learning from scratch. Finally, six self-supervised methods underperform ResNet50 learning from scratch. These results also highlight the importance of the pretext tasks for self-supervised learning, and the performance can vary significantly with these tasks for the same backbone. In the context of clinical practice, the self-supervised pre-training provides a good initialization for fine-tuning the downstream task of PE slice-level classification. However, an unfavorable pretext task leads to inferior performance on the downstream task compared to scratch training. It is believed that a good pretext task based on large-scale PE detection will provide a stronger initialization for robust and efficient slice-level PE classification. On the other hand, SSL can be useful in the clinical practice of PE slice-level classification due to its ability to learn from unlabeled data. In a clinical setting, obtaining annotated data for PE classification can be challenging and time-consuming, and SSL can leverage the large amounts of available unlabeled data to learn a representation that can be used for classification tasks.

The details and comparison of the backbones and the self-supervised methods follow:

### Backbone Architectures:

ResNet18, ResNet50 and ResNet101 (He et al., 2016): One way to improve an architecture is to add more layers and make it deeper. Unfortunately, increasing the depth of a network cannot be simply accomplished by stacking layers together because such methodology introduces a problem called vanishing gradient. Moreover, the performance may become saturated or decreased overtime. The main idea behind ResNet is to have an identity shortcut connection which skips one or more layers. According to the inventors, stacking layers should not decrease the performance of the network. The residual block allows the network to have identity mapping connections which prevents vanishing gradient. The inventors presented several versions of ResNet models including ResNet18, ResNet34, ResNet50 and ResNet101. The numbers indicate how many layers exist within the architecture. The more layers represent a deeper network thus increasing the trainable parameters.

ResNext50 (Xie et al., 2017): In ResNext50, the authors introduced a new dimension C, which is called Cardinality. Cardinality controls the size of the set of transformations in addition to the dimensions of depth and width. The inventors contend that increasing cardinality is more effective than going deeper or wider in terms of layers and used this architecture in the ILSVRC 2016 Classification Competition and won 2nd place. ResNext50 has a similar number of parameters for training and can boost performance, and could achieve nearly equivalent performance to ResNet101 although ResNet101 has deeper layers.

DenseNet121 (Huang et al., 2017): Increasing the depth of a network results in performance improvement. However, a problem arises when the network is too deep. As a result, the path between input and output becomes too long which introduces a well-known issue called vanishing gradient. DenseNets simply redesign the connectivity pattern of the network so that the maximum information is flown. The main idea is to connect every layer directly with each other layer in a feed-forward fashion. For each layer, the feature-maps of all preceding layers are used as inputs, and its own feature-maps are used as inputs into all subsequent layers. According to the embodiments, the advantages of using DenseNet is that this approach alleviates the vanishing gradient problem, strengthens feature propagation, encourages feature reuse, and substantially reduces the number of parameters.

Xception (Chollet, 2017): Xception network architecture was built on top of Inception-v3. It is also known as an extreme version of the Inception module, with a modified depthwise separable convolution, superior to Inception-v3. The original depthwise separable convolution approach is to perform depth-wise convolution first, followed by a pointwise convolution. Here, depthwise convolution is the channel-wise partial convolution, and pointwise convolution is the 1×1 convolution to change the dimension. This strategy is modified for Xception architecture. In Xception, the depthwise separable convolution performs 1×1 pointwise convolution first and then the channel-wise spatial convolution. Moreover, Xception and Inception-v3 has the same number of parameters. The Xception architecture slightly outperforms Inception-v3 on the ImageNet dataset and significantly outperforms Inception-v3 on a larger image classification dataset comprised of 350 million images and 17,000 classes.

DRN-A-50 (Yu et al., 2017): Typically in an image classification task, the Convolutional Neural Network progressively reduces resolution until the image is represented by tiny feature-maps in which the spatial structure of the scene is not quite visible. This kind of spatial structure loss can hamper image classification accuracy as well as complicate the transfer of the model to a downstream task. This architecture introduces dilation, which increases the resolutions of the feature-maps without reducing the receptive field of individual neurons. Dilated residual networks (DRNs) can outperform their non-dilated counterparts in image classification task. This strategy does not increase the model's depth or complexity. As a result, the number of parameters remains constant relative to their non-dilated counterparts.

SeNet154 (Hu et al., 2018b): The convolution operator enables networks to construct informative features by fusing both spatial and channel-wise information within local receptive fields at each layer. This work focused on a channel-wise relationship and proposed a novel architectural unit called Squeeze-and-Excitation (SE) block. This SE block adaptively re-calibrates channel-wise feature responses by explicitly modeling inter-dependencies between channels. These blocks can also be stacked together to form a network architecture (SeNet154) that is generalized yet effective across different datasets. SeNet154 is one of the superior models used in ILSVRC 2017 Image Classification Challenge and won 1st place.

SeResNet50, SeResNet101, SeResNext50 and SeXception (Hu et al., 2018b): The structure of the Squeeze-and-Excitation (SE) block is very simple and can be added to any state-of-the-art architectures by replacing components with their SE counterparts. SE blocks are also computationally lightweight and impose only a slight increase in model complexity and computational burden. Therefore, SE blocks were added to the ResNet50 and ResNext50 models when designing the new version. The pre-trained weights for SeResNet50 and SeResNext50 already exist whereas SeXception is not available publicly. By adding SE blocks, the SeXception architecture was created and trained on the ImageNet dataset to achieve the pre-trained weights. Subsequently, the pre-trained weights were used for transfer learning schemes.

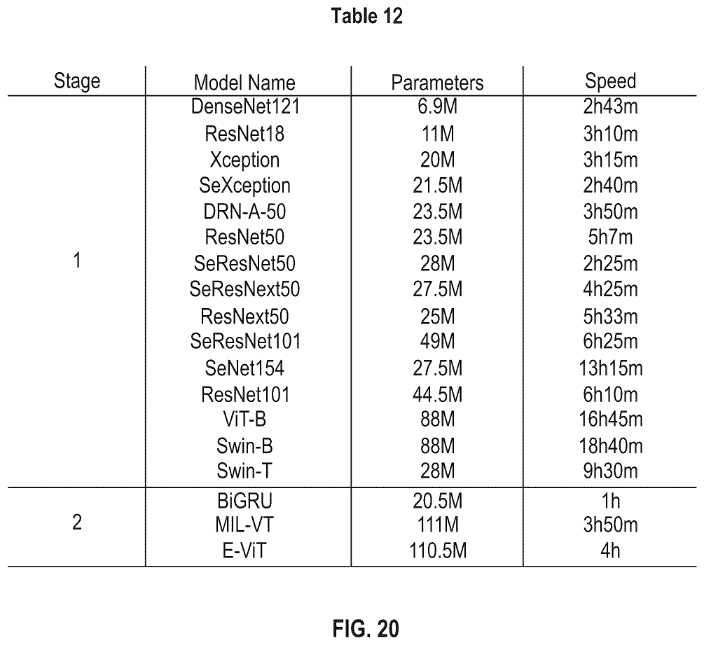

FIG. 20 displays table 12 which provides an overview of the parameter size and training speed for all models used in researching embodiments of the invention.

### Self Supervised Methods:

InsDis (Wu et al., 2018): InsDis trains a non-parametric classifier to distinguish between individual instance classes based on NCE (noise-contrastive estimation) (Gutmann and Hyvärinen, 2010). Moreover, each instance of an image functions as its own distinct class for the classifier. InsDis also introduces a feature memory bank to maintain a large number of noise samples (referring to negative samples), thus avoiding exhaustive feature computing.

MoCo-v1 (He et al., 2020), MoCo-v2 (Chen et al., 2020c) and MoCo-v3 (Chen et al., 2021): MoCo-v1 uses data augmentation to create two views of a same image X, referred to as positive samples. Similar to InsDis, images other than X are defined as negative samples and are stored in a memory bank. Moreover, to ensure the consistency of negative samples, a momentum encoder is introduced as the samples evolve during the training process. Basically, the proposed method aims to increase the similarity between positive samples while decreasing the similarity between negative samples. On the other hand, MoCo-v2 works similarly by adding a non-linear projection head, few more augmentations, cosine decay schedule, and a longer training time.

SimCLR-v1 (Chen et al., 2020a) and SimCLR-v2 (Chen et al., 2020b): The key idea of SimCLR-v1 is similar to MoCo yet they were proposed independently. Here, SimCLR-v1 is trained in an end-to-end fashion with larger batch sizes instead of using special network architectures (a momentum encoder) or a memory bank. Within each batch, the negative samples are generated on the fly. However, SimCLR-v2 optimizes the previous version by increasing the capacity of the projection head and incorporating the memory mechanism from MoCo to provide more meaningful negative samples.

BYOL (Grill et al., 2020): MoCo and SimCLR methods rely mainly on a large number of negative samples and require either a large memory bank or a large batch size. On the other hand, BYOL replaces the use of negative pairs by adding an online encoder, target encoder, and a predictor after the projector in the online encoder. Both the target encoder and online encoder computes features. The key idea is to maximize the agreement between the target encoder's features and prediction from the online encoder. To prevent the collapsing problem, the target encoder is updated by the momentum mechanism.

PIRL (Misra and Maaten, 2020): Both InsDis and MoCo take advantage of using instance discrimination. However, PIRL adapts the Jigsaw and Rotation as proxy tasks. Here, the positive samples are generated by applying Jigsaw shuffling or rotating by {0°, 90°, 180°, 270°}. Following InsDis, PIRL uses Noise-Contrastive Estimation (NCE) as loss function and a memory bank.

DeepCluster-v2 (Caron et al., 2021a): DeepCluster (Caron et al., 2018) uses two phases to learn features. First, it uses self-labeling, where pseudo labels are generated by clustering data points using prior representation, yielding cluster indexes for each sample. Secondly, it uses feature-learning, where each sample's cluster index is used as a classification target to train a model. Until the model is converged, the two phases mentioned are repeated. The DeepCluster-v2 minimizes the distance between each sample and the corresponding cluster centroid. DeepCluster-v2 also uses stronger data augmentation, an MLP projection head, a cosine decay schedule, and multi-cropping to improve the representation learning.

SeLa-v2 (Caron et al., 2021a): SeLa also requires two-phase training (self-labeling and feature-learning). SeLa focuses on self-labeling as an optimal transport problem and solves it using a Sinkhorn-Knopp algorithm. SeLa-v2 also uses stronger data augmentation, an MLP projection head, a cosine decay schedule, and multi-cropping to improve the representation learning.

PCL-v1 and PCL-v2 (Asano et al., 2020): PCL-v1 aims to bridge contrastive learning with clustering. PCL-v1 adopts the same architecture as MoCo, including an online encoder and a momentum encoder. Following clustering-based feature learning, PCL-v1 also uses two phases (self-labeling and feature-learning). The features obtained from the momentum encoder are clustered in the self-labeling phase. On the other hand, PCL-v1 generalizes the NCE loss to ProtoNCE loss instead of classifying the cluster index with regular cross-entropy. This was done in PCL-v2 as an improvement step.

SwAV (Caron et al., 2021a): SwAV uses both contrastive learning as well as clustering techniques. For each data sample, SwAV calculates cluster assignments (codes) with the help of a Sinkhorn-Knopp algorithm. Moreover, SwAV works on-line performing assignments at the batch level instead of epoch level.

InfoMin (Tian et al., 2020): InfoMin suggested that for contrastive learning, the optimal views depend upon the downstream task. For optimal selection, the mutual information between the views should be minimized while preserving the task-specific information.

Barlow Twins (Zbontar et al., 2021b): The Barlow Twins consists of two identical networks fed with two distorted versions of the input sample. The network is trained such that the cross-correlation matrix between the two resultant embedding vectors is close to the identity. A regularization term is also included in the objective function to minimize redundancy between embedding vectors' components.

SimSiam (Chen and He, 2021) SimSiam uses a simple Siamese network to learn meaningful representation. Unlike the standard self-supervised methods, SimSiam does not use (i) negative sample pairs, (ii) large batches, and (iii) momentum encoders.

DINO (Caron et al., 2021b) DINO can be interpreted as Knowledge Distillation with no labels. The transformed inputs are passed through both a teacher and student model. The network is trained to minimize the difference between the teacher and student outputs using cross-entropy loss.

OBoW (Gidaris et al., 2021) OboW is a teacher-student learning paradigm that uses self-supervised training to learn bag-of-visual-words (BoW) representation from images. The teacher model generates BoW targets, and the student model learns the BoW representation for the perturbed input image.

CLSA (Wang and Qi, 2021) As discussed herein, the inventors propose a strong augmentation, which combines 14 types of augmentations: ShearX/Y, TranslateX/Y, Rotate, AutoContrast, Invert, Equalize, Solarize, Posterize, Contrast, Color, Brightness, and Sharpness. Instead of directly applying strong augmentation while training, CLSA proposes to minimize the distribution divergence between strongly and weakly augmented images.

### 5.1.3. Transformer-Based Models Demonstrate Comparable Performance With Slower Convergence Speed

FIG. 11 displays Table 3. For slice-level PE classification task, ViT models exhibit inferior performance when compared to CNNs. Conversely, Swin Transformers demonstrate performance on par with SeXception. It is worth noting that both ViT and Swin Transformer models exhibit a tendency to converge at a slower rate. The effectiveness of using ViT and Swin Transformer backbones for this task was evaluated under various settings. Initiating these models with ImageNet pre-training significantly enhances performance, similar to CNNs. Additionally, initializing the weights from a self-supervised pre-trained model further improves performance. Table 3 provides compelling evidence that the Swin Transformer (Swin-B), which was pre-trained using the SimMIM self-supervised approach, outperforms other transformer-based models, attaining an impressive AUC score of 96.46, even with a larger image size of 448×448. Notably, Swin-T, pre-trained with ImageNet1k, achieves a commendable AUC score of 96.30, coming remarkably close to the performance of the standalone CNN architecture (SeXception). However, it is important to note that fine-tuning transformer-based models required substantially more time and a greater number of training epochs to reach convergence. Also, the performance of the transformer-based models varies significantly with the training set size. As the transformer-based models are trained using the image patches, two approaches are followed to expand the training set:

The patch size is decreased for a given image size, effectively increasing the total number of patches.

The image size is increased for a given patch size, thus enhancing the total patch count.

The results demonstrate the crucial role of initialization as pre-training for transformer-based models, highlighting its significance (Ma et al., 2022). In contrast to CNNs, the number of parameters was large for transformer-based models and it took more than 16 hours to finish one epoch of training. Although there are a huge number of slices, CNNs took only about 3 hours to complete one epoch of training.

### 5.1.4. Squeeze and Excitation (SE) Block Further Enhances CNN Performance

Despite fewer parameters compared with many other architectures, SeXception provides an optimal average AUC of 96.34±0.09. Thus, the SE blocks are parameter-efficient and have led to performance improvements for a variety of CNN architectures, such as ResNet50, ResNet101, ResNext50.

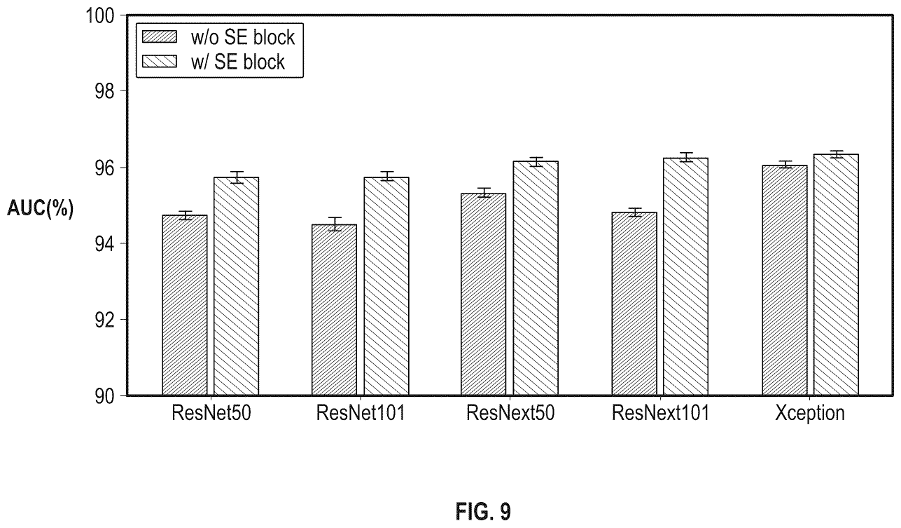

ResNext101, and Xception (FIG. 9). A performance gain was observed with the help of SE block. Note that all architectures are pre-trained from ImageNet. This observation is consistent with Hu et al. (2018a), which infer SE blocks capture feature dependencies in the channels to improve the performance. Here, SE blocks in a CNN enhance PE detection by finding relationships between consecutive CT slices, thereby improving the model's ability to identify and classify PEs with higher accuracy and efficiency. Additionally, the SE blocks facilitate learning more discriminative features and improve the handling of large variability in medical images, resulting in enhanced accuracy and robustness in PE slice-level classification for CTPA data. Consequently, the utilization of SE blocks is deemed a valuable tool in clinical practice.

### 5.2. Exam-Level Diagnosis

### 5.2.1. Sequence Models Benefit From More Comprehensive Slice-Level Embeddings

FIG. 12 displays Table 4. The embeddings extracted by the slice-level classification model are passed through BiGRU for exam-level diagnosis. However, no model performs optimally across all exam-level labels. The mean AUC over 10 runs was reported and the optimal results bolded for each label. The ensemble of Xception, SeXception and SeResNext50 achieves a significant improvement (p=5.94E-13) over the previous state of the art. Table 4 summarizes the results of the sequential model learning (explained in § 4.2.1) for exam-level diagnosis. Although the combination of SeResNext50, Xception and SeXception performs optimally for slice-level classification (see Table 4), this is not the case for exam-level diagnosis. As a standalone CNN backbone, Xception achieves the best mean AUC score across nine exam-level labels gaining 1.05% compared with the previous state of the art (SeResNext50). The exam-level diagnosis result is combined by taking the average from Xception and SeXception, SeResNext50 and SeXception, and SeResNext50 and Xception and SeXception. It was observed that Xception and SeXception achieve a significant gain of 1.21% (p=5.94E-13) over the state of the art. Subsequently, more extensive slice-level embeddings derived from Swin-B and Swin-T were harnessed to enhance the exam-level diagnosis (Table 5). So far, Swin-T, acting as the backbone, has established itself as the leading performer for exam-level PE diagnosis, demonstrating an impressive AUC of 90.11 and achieving a notable improvement of 0.83 (p=6.81E-14) compared to the ensemble of Xception and SeXception.

### 5.2.2. E-ViT Further Exceeds Sequence Models

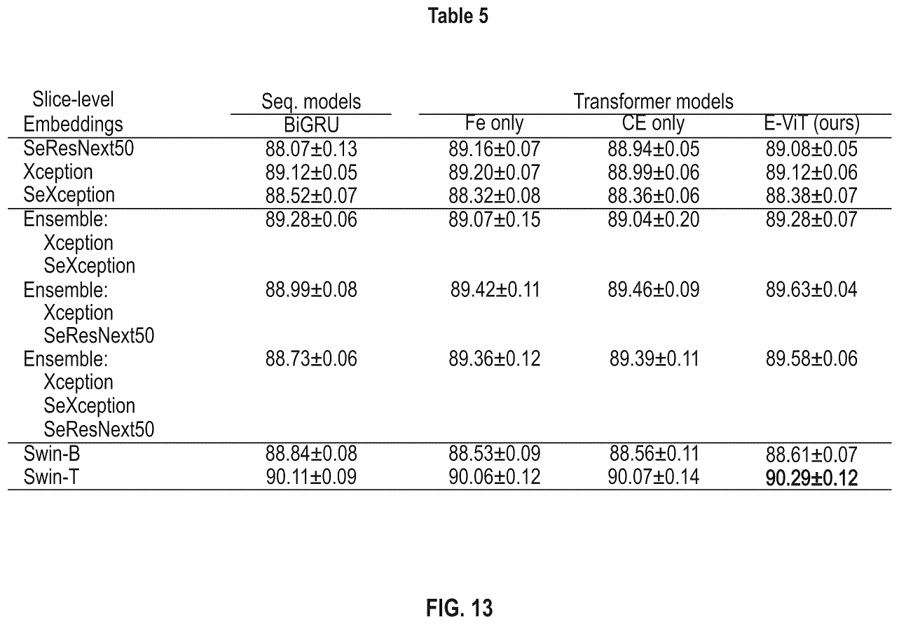

FIG. 13 displays Table 5. The extracted embeddings from the slice-level classification were used as input for E-ViT for the exam-level diagnosis. FE and CE stand for exam-level embedding and class embedding from ViT, respectively. E-ViT represents the proposed framework where the loss is calculated by taking the average of FE and CE while training. At the inference stage, the output of FE and CE are averaged to obtain the exam-level prediction. Each experiment was conducted 10 times and the mean AUC reported for each of the nine exam-level diagnosis with standard deviation. The best result is highlighted in bold. Table 5 demonstrates the mean AUC for all the exam-level predictions with sequence model and the proposed Transformer-based model. In the evaluation, utilizing only class embedding for exam-level diagnosis results in performance comparable to the sequence model. Conversely, utilizing only the exam-level embedding from the Transformer encoder leads to superior performance, surpassing both the sequence model and E-ViT with class embedding alone. The integration of class embedding and exam-level embedding in the E-ViT framework results in even further improved performance for exam-level diagnosis. These observations are consistent across slice-level embeddings extracted from three different backbones (i.e., SeResNext50, Xception and SeXception). When the slice-level embeddings are extracted from SeResNext50 and used as inputs, E-ViT outperforms sequence model by an AUC gain of 1.01%. The performance achieved by combining two or three architectures together were analyzed by simply taking the mean prediction of each exam-level across slice-level embeddings extracted from different backbones. The best AUC performance (89.63) is achieved from the ensemble of Xception and SeResNext50, gaining 1.56% over the first-place solution with a statistical significance of 2.19E-11 when using CNN architecture as backbone. Slice-level embeddings from Swin-B and Swin-T were used for E-ViT for the task of exam-level PE diagnosis. Notably, the findings revealed that the pairing of Swin-T as the backbone with E-ViT yielded the most impressive performance, achieving an AUC of 90.29 for exam-level diagnosis. While the combination of Swin-T and BiGRU exhibited similar results, E-ViT outperformed it significantly (p=8.61E-4).

In clinical practice, the number of slices in a CT scan can vary greatly between patients, making it challenging to use traditional models that require a fixed number of inputs. The proposed Transformer-based model, E-ViT, is designed to tackle this issue by adapting to varying numbers of slices without losing any expert annotations. This is an important aspect for its use in clinical practice, as it allows for a more efficient and accurate diagnosis of PE. The following discussion presents ablation studies on E-ViT.

Position embedding decreases performance. Experiments were conducted to analyze the significance of position embedding in E-ViT during exam-level PE diagnosis. Position embedding is a learnable parameter added to patches in the original ViT architecture to retain positional information. Slice-level embeddings rather than image patches are used as inputs to E-ViT. Table 6 shows that adding position embedding to slice-level embeddings decreases the performance of the model. Unlike photographic images where each patch constitutes distinctive visual features, consecutive slices in a CTPA scan are similar to one another. It is hypothesized that similar consecutive slices leads to similar embeddings, thus making it complex for E-ViT to learn the position embedding. FIG. 14 displays Table 6. In the evaluation of the E-ViT framework, the impact of position embedding on the exam-level diagnosis was investigated. The disclosed embodiments combine class embedding and exam-level embedding, and utilize the SeResNext50 model to obtain the slice-level embeddings. The results, presented in Table 6, demonstrate a decrease in mean AUC score with the addition of position embedding, regardless of whether it is utilized together with class embedding, exam-level embedding, or as a standalone component. The experiments were repeated 10 times to calculate the standard deviation.

Benefits of combining class and exam-level embeddings. Original ViT only uses the class embedding for classification tasks and ignores all other features extracted from the Transformer encoder. The experiments are conducted to analyze the effectiveness of class embedding, exam-level embeddings, and both the embeddings used simultaneously for exam-level PE diagnosis. Table 5 shows that individual class embedding performs inferior to both exam-level embedding, and the combination of both embeddings for all models. Based on this observation, it is hypothesized that class embedding cannot provide rich semantic features from similar-appearing medical slices. Exam-level embedding provide a better feature representation; moreover, fusing exam-level embedding with class embedding provides the best results.

Comparison with MIL-VT. Multiple Instance Learning Enhanced Vision Transformer (MIL-VT) (Yu et al., 2021) introduced a multiple instance learning (MIL) head to the original ViT to enhance the learned features. The embeddings from the MIL head were combined with class embedding for final classification. Similarly, E-ViT also uses both class embedding and exam-level embedding from the Transformer encoder, but it is different from MIL-VT regarding the architecture design of the MIL head. MIL-VT uses lower dimension embedding with an attention aggregator on the features extracted from the Transformer encoder, whereas E-ViT uses attention weighted average pooling with max pooling. During training, the average loss of each branch (class embedding and exam-level embedding) is taken, whereas during testing, the mean predictions from each branch are computed. FIG. 15 displays Table 7 which shows E-ViT significantly outperforms MIL-VT by 0.28% AUC for exam-level diagnosis. It is also observed that position embedding continues to decrease the performance of both E-ViT and MIL-VT.

### 5.3. False Positive Reduction

### 5.3.1. 3D Data Offer Higher Performance Than 2D and 2.5D Data

While it may appear that using 3D models to handle 3D volumetric data is a reasonable choice, it comes with a high computing cost and a risk for overfitting (Zhou et al., 2021b). As a result, various alternative methodologies for reformatting 3D applications into 2D solutions have been presented. Regular 2D inputs were constructed by extracting neighboring axial slices (referred to as 2D slice-based input) by Ben-Cohen et al. (2016) and Sun et al. (2017). These 2D reformatted solutions create large amounts of data and take advantage of 2D models pre-trained on ImageNet. However, 2D solutions unavoidably compromise the rich spatial information in 3D volumetric data and the large capacity of 3D models. Alternatively, a more advanced technique, as detailed by Prasoon et al. (2013) and Roth et al. (2014, 2015), is to extract axial, coronal, and sagittal slices from volumetric data (referred to as 2.5D orthogonal input).

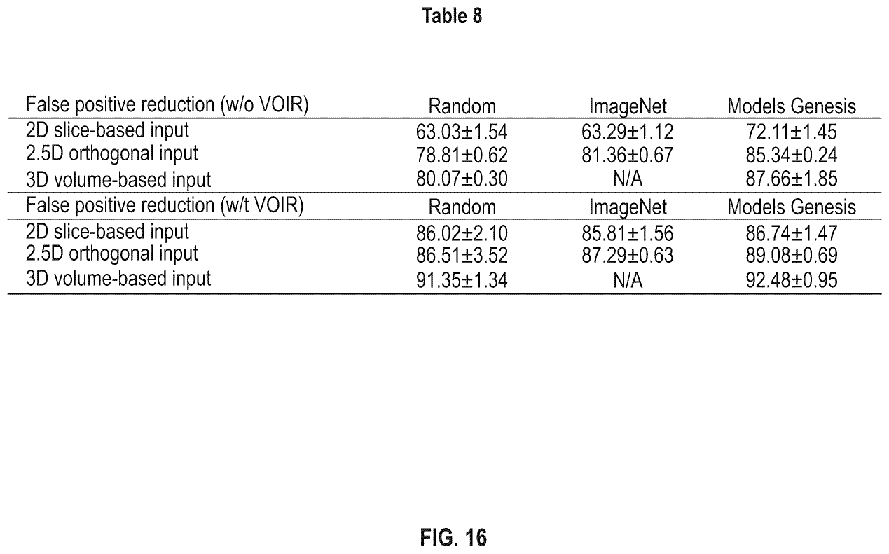

FIG. 16 displays Table 8, in which vessel-oriented image representation (VOIR) (Tajbakhsh et al., 2019) is evaluated in comparison with 2D, 2.5D, and 3D solutions for the task of reducing PE false positives. All the results in the table are candidate-level AUC, including the mean and standard deviation across 10 trials. Table 8 compares 2D slice-based, 2.5D orthogonal, and 3D volume-based approaches as different image dimensions for PE false positive reduction. According to analyses, 3D volume-based data consistently outperforms 2D and 2.5D data because it contains significantly more information. When the model is trained from scratch, 3D volume-based data outperforms 2.5D orthogonal data in both VOIR and non-VOIR scenarios (78.81% vs. 80.07% and 86.51% vs. 91.35%). Here, the 3D volume-based data significantly outperforms the 2D slice-based and 2.5D orthogonal approaches, with p-values of 7.94E-13 for non-VOIR and 2.19E-10 for VOIR scenarios, respectively. These results indicate that the use of 3D data in CTPA can lead to a reduction in false positive results compared to 2D or 2.5D data. This is because 3D data provides a more comprehensive and detailed view of the blood vessels in the lungs, which can aid in the accurate identification and differentiation of true positives from false positives. Overall, the use of 3D data in CTPA can improve the accuracy and reliability of the results, leading to better patient care in the clinical context.

### 5.3.2. VOIR is More Informative Than the Standard Image Representation, Boosting Performance Across Image Dimensions

Developing a computer-aided diagnosis (CAD) system that can accurately detect pulmonary embolism (PE) while maintaining a clinically acceptable level of false positives is highly desirable (Tajbakhsh et al., 2016). CTPA is the primary imaging modality used to diagnose PE, and appropriate pre-processing of these images is necessary to ensure accurate detection and reduce false positives. One approach to improve the performance of the model is through the use of vessel-oriented image representation (VOIR) (Tajbakhsh et al., 2019), which promotes consistency in the data and has been shown to be effective for PE detection. However, previous implementations of VOIR were limited to a 2D representation and did not fully exploit the 3D information available in CTPA images. Embodiments extend VOIR to a 3D form (§ 4.3) and evaluate its performance under various settings to improve the CAD system's ability to accurately detect PE and reduce false positives for better clinical outcomes.

Table 8 summarizes the results with VOIR and non-VOIR using 2D, 2.5D, and 3D input data as different image dimensions. The AUC is higher with VOIR data in all image dimensions (2D, 2.5D, and 3D). As discussed in § 5.3.1, although 2.5D data is expected to be more valuable than 2D data (Zhou, 2021), the results show that 2D data with VOIR significantly outperforms 2.5D data non-VOIR by an AUC gain of 7.21%. Similarly, 2D slice-based VOIR data outperforms 3D volume-based non-VOIR data (5.95% gain), even though 3D data contains significantly more information. These results show that vessel-oriented image representation is more informative than the standard image representation for reducing PE false positives.

### 5.3.3. Same Domain Transfer Learning With Self-Supervised Pre-Training Enhances Performance Across Image Representations and Dimensions

One of the most commonly utilized paradigms in deep learning for medical image analysis is transfer learning from photographic images to medical images. Medical images, such as those used in radiology and diagnostic imaging, are distinct from photographic images in that they are often monochromatic and feature consistent anatomical structures. These characteristics can make medical images more challenging to interpret and analyze (Hosseinzadeh Taher et al., 2021). Therefore, same-domain transfer learning should be applied since a smaller domain gap makes the learned image representation more effective for target tasks (Zhou et al., 2021b). Models Genesis (Zhou et al., 2019), a self-supervised method, were developed to mitigate the domain gap. Specifically, Models Genesis were pre-trained on LUNA16 (Setio et al., 2016), a chest CT dataset that shares similar body parts and imaging modality as the PE-CAD dataset. Models Genesis with ResNet18 was used as the backbone for the task of PE false positive reductions. The recently published 3D self-supervised approaches (Guo et al., 2022) were evaluated, which were pre-trained on the LUNA16 dataset, to perform the same task. The inventors anticipate improved performance in image representation (non-VOIR vs. VOIR) and dimensions (2D vs. 2.5D vs. 3D) compared to supervised pre-trained models due to their self-supervised nature.

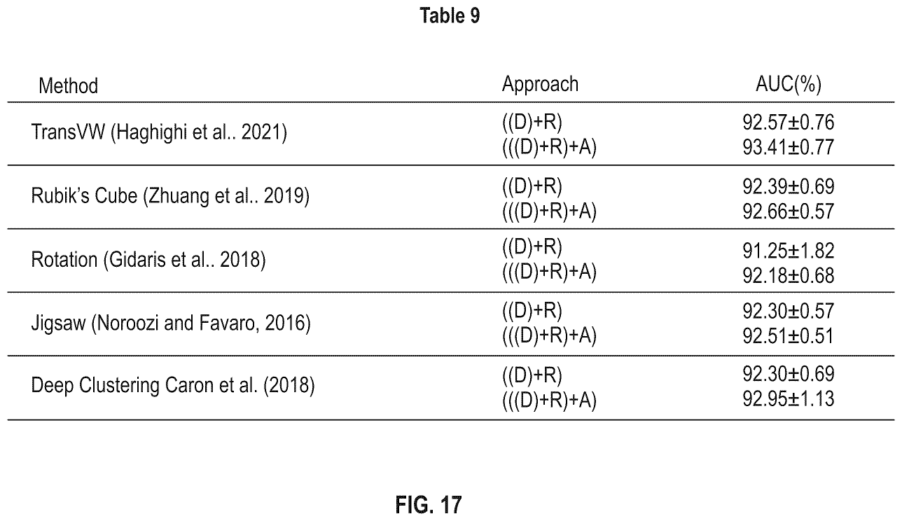

According to Table 8, weights initialized from Models Genesis consistently outperform the ImageNet counterpart. For 2D slice-based non-VOIR data, Models Genesis initialization achieves an AUC of 72.11%±1.45 compared to 63.29%±1.54 with ImageNet initialization, achieving a significant gain of 8.82% (p-value=2.63E-11). Similarly, for 2.5D orthogonal non-VOIR inputs, the gain is 2.98% with a p-value of 1.34E-9. A similar performance is observed for VOIR data, with a gain of 0.93% and 1.79% for 2D and 2.5D input data, respectively. Hence, self-supervised pre-training with Models Genesis enhances performance across image representations and dimensions. The results demonstrate the advantages of using the same domain transfer learning in medical image analysis. This approach involves utilizing models trained on similar medical image data to perform a new task. For instance, it was found that transfer learning within the same domain is advantageous in medical image analysis as it allows the model to utilize its previously learned features to efficiently adapt to a new task (Zhou et al., 2019). As a result, same domain transfer learning can significantly enhance the accuracy and efficiency of medical image analysis in the clinical context, ultimately leading to better patient care and outcomes. Furthermore, results were demonstrated by conducting an evaluation of the latest 3D self-supervised pre-trained models on the 3D VOIR dataset. According to Table 9, the TransVW model with the pre-training strategy of (((D)+R)+A) performs best in reducing false positives on 3D VOIR data, achieving an AUC of 93.41%. Here, D represents the discriminative encoder, R is the restorative decoder, and A the adversary encoder. FIG. 17 displays Table 9. The performance of recent 3D self-supervised methods as outlined in (Guo et al., 2022) on the 3D VOIR dataset were compared, focusing on the task of reducing false positives. The results are based on ten runs and are presented as mean and standard deviation values. The study demonstrates that using a self-supervised pre-trained model with the same domain transfer learning can significantly improve false positive reduction performance on the 3D VOIR dataset.

### 6. Conclusion

For the RSNA PE dataset, the existing first-place solution utilizes SeResNext50 for slice-level classification and bidirectional GRU for exam-level diagnosis. An optimized approach achieves a significant increase in the AUC for both slice-level classification and exam-level diagnosis. Through rigorous analysis, it was determined that the optimal architecture for slice-level classification is an ensemble of Xception, SeXception and SeResNext50, resulting in an AUC gain of 0.62%. To further improve exam-level diagnosis performance, a novel E-ViT model offers a significant performance gain of 2.22%. Experiments using in-house PE-CAD dataset have shown that vessel-oriented image representation, as a pre-processing step, is important for reducing false positives, and has a considerable positive impact on performance across image dimensions, boosting the performance from 63.03% to 86.02% in 2D, from 78.81% to 86.51% in 2.5D, and from 80.07% to 91.35% in 3D. Moreover, the use of Models Genesis in pre-training has resulted in a significant improvement in performance across image representations and dimensions: from 72.11% to 86.74% in 2D, 85.34% to 89.08% in 2.5D. Finally, using the self-supervised TransVW model with (((D) +R) +A) on the 3D VOIR data significantly improves false positive reduction performance, achieving an outstanding AUC of 93.41%.

Embodiments of the invention contemplate a machine or system within which embodiments may operate, be installed, integrated, or configured. In accordance with one embodiment, the system includes at least a processor and a memory therein to execute instructions including implementing any application code to perform any one or more of the methodologies discussed herein. Such a system may communicatively interface with and cooperatively execute with the benefit of remote systems, such as a user device sending instructions and data, a user device to receive output from the system.

A bus interfaces various components of the system amongst each other, with any other peripheral(s) of the system, and with external components such as external network elements, other machines, client devices, cloud computing services, etc. Communications may further include communicating with external devices via a network interface over a LAN, WAN, or the public Internet.

In alternative embodiments, the system may be connected (e.g., networked) to other machines in a Local Area Network (LAN), an intranet, an extranet, or the public Internet. The machine may operate in the capacity of a server or a client machine in a client-server network environment, as a peer machine in a peer-to-peer (or distributed) network environment, as a server or series of servers within an on-demand service environment. Certain embodiments of the machine may be in the form of a personal computer (PC), a tablet PC, a set top box (STB), a Personal Digital Assistant (PDA), a cellular telephone, a web appliance, a server, a network router, switch or bridge, computing system, or any machine capable of executing a set of instructions (sequential or otherwise) that specify and mandate the specifically configured actions to be taken by that machine pursuant to stored instructions. Further, the term “machine” shall also be taken to include any collection of machines (e.g., computers) that individually or jointly execute a set (or multiple sets) of instructions to perform any one or more of the methodologies discussed herein.

An exemplary computer system includes a processor, a main memory (e.g., read-only memory (ROM), flash memory, dynamic random access memory (DRAM) such as synchronous DRAM (SDRAM) or Rambus DRAM (RDRAM), etc., static memory such as flash memory, static random access memory (SRAM), volatile but high-data rate RAM, etc.), and a secondary memory (e.g., a persistent storage device including hard disk drives and a persistent database and/or a multi-tenant database implementation), which communicate with each other via a bus. Main memory includes code that implements the three branches of the SSL framework described herein, namely, the localizability branch, the composability branch, and the decomposability branch.

The processor represents one or more specialized and specifically configured processing devices such as a microprocessor, central processing unit, or the like. More particularly, the processor may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processor may also be one or more special-purpose processing devices such as an application-specific integrated circuit (ASIC), a field programmable gate array (FPGA), a digital signal processor (DSP), network processor, or the like. The processor is configured to execute processing logic for performing the operations and functionality discussed herein.

The system may further include a network interface card. The system also may include a user interface (such as a video display unit, a liquid crystal display, etc.), an alphanumeric input device (e.g., a keyboard), a cursor control device (e.g., a mouse), and a signal generation device (e.g., an integrated speaker). According to an embodiment of the system, the user interface communicably interfaces with a user client device remote from the system and communicatively interfaces with the system via a public Internet.

The system may further include peripheral devices (e.g., wireless or wired communication devices, memory devices, storage devices, audio processing devices, video processing devices, etc.).

A secondary memory may include a non-transitory machine-readable storage medium or a non-transitory computer readable storage medium or a non-transitory machine-accessible storage medium on which is stored one or more sets of instructions (e.g., software) embodying any one or more of the methodologies or functions described herein. The software may also reside, completely or at least partially, within the main memory and/or within the processor during execution thereof by the system, the main memory and the processor also constituting machine-readable storage media. The software may further be transmitted or received over a network via the network interface card.

In addition to various hardware components depicted in the figures and described herein, embodiments further include various operations which are described herein. The operations described in accordance with such embodiments may be performed by hardware components or may be embodied in machine-executable instructions, which may be used to cause a specialized and special-purpose processor having been programmed with the instructions to perform the operations described herein. Alternatively, the operations may be performed by a combination of hardware and software. In such a way, the embodiments of the invention provide a technical solution to a technical problem.

Embodiments also relate to an apparatus for performing the operations disclosed herein. This apparatus may be specially constructed for the required purposes, or it may be a special purpose computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions, each coupled to a computer system bus.

While the algorithms and displays presented herein are not inherently related to any particular computer or other apparatus, they are specially configured and implemented via customized and specialized computing hardware which is specifically adapted to more effectively execute the novel algorithms and displays which are described in greater detail herein. Various customizable and special purpose systems may be utilized in conjunction with specially configured programs in accordance with the teachings herein, or it may prove convenient, in certain instances, to construct a more specialized apparatus to perform the required method steps. The required structure for a variety of these systems will appear as set forth in the description. In addition, embodiments are not described with reference to any particular programming language. It will be appreciated that a variety of programming languages may be used to implement the teachings of the embodiments as described herein.

Embodiments may be provided as a computer program product, or software, that may include a machine-readable medium having stored thereon instructions, which may be used to program a computer system (or other electronic devices) to perform a process according to the disclosed embodiments. A machine-readable medium includes any mechanism for storing or transmitting information in a form readable by a machine (e.g., a computer). For example, a machine-readable (e.g., computer-readable) medium includes a machine (e.g., a computer) readable storage medium (e.g., read only memory (“ROM”), random access memory (“RAM”), magnetic disk storage media, optical storage media, flash memory devices, etc.), a machine (e.g., computer) readable transmission medium (electrical, optical, acoustical), etc.

Any of the disclosed embodiments may be used alone or together with one another in any combination. Although various embodiments may have been partially motivated by deficiencies with conventional techniques and approaches, some of which are described or alluded to within the specification, the embodiments need not necessarily address or solve any of these deficiencies, but rather, may address only some of the deficiencies, address none of the deficiencies, or be directed toward different deficiencies and problems which are not directly discussed.

While the subject matter disclosed herein has been described by way of example and in terms of the specific embodiments, it is to be understood that the claimed embodiments are not limited to the explicitly enumerated embodiments disclosed. To the contrary, the disclosure is intended to cover various modifications and similar arrangements as are apparent to those skilled in the art. Therefore, the scope of the appended claims is to be accorded the broadest interpretation to encompass all such modifications and similar arrangements. It is to be understood that the above description is intended to be illustrative, and not restrictive. Many other embodiments will be apparent to those of skill in the art upon reading and understanding the above description. The scope of the disclosed subject matter is therefore to be determined in reference to the appended claims, along with the full scope of equivalents to which such claims are entitled.

What is claimed is:

1. A system comprising:
a memory to store instructions;
a processor to execute the instructions stored in the memory to perform the following operations:
receiving a plurality of Computed Tomography Pulmonary Angiography (CTPA) exams (hereinafter the “exams”) as input image data, each exam in the plurality of exams comprising a varying plurality of individual images (hereinafter the “slices”);
annotating each of the slices with one of a pulmonary embolism (PE) present label or PE absent label;
annotating each of the exams with one of a plurality of labels each indicating a different PE state and location;
performing a slice-level PE classification to determine the presence or absence of PE for each of the slices; and
performing and outputting, via an Embedding-based Vision Transformer (E-ViT), an exam-level diagnosis using the slice-level classifications.

2. The system of claim 1 wherein the plurality of exams comprises one of the Radiological Society of North America (RSNA) Pulmonary Embolism (PE) CT dataset and the Computer Aided Diagnosis-Pulmonary Embolism (CAD-PE) Challenge dataset, and an in-house PE-CAD dataset.

3. The system of claim 2, wherein performing slice-level PE classification to determine the presence or absence of PE for each of the slices, comprises pre-processing steps of lung localization to focus on a region of interest in the slices, and windowing to highlight pixel intensities within a range of 100-700 Hounsfield Units.

4. The system of claim 2, wherein the plurality of exams comprises the RSNA PE CT dataset, and wherein performing slice-level PE classification to determine the presence or absence of PE for each of the slices comprises performing slice-level classification using an ensemble of CNN-based architectures including Xception, SeXception and SeResNext50.

5. The system of claim 2, wherein the plurality of exams comprises the CAD-PE Challenge dataset, and wherein performing slice-level PE classification to determine the presence or absence of PE for each of the slices comprises a pre-processing step of representing the input image data using a three-dimensional (3D) vessel-oriented image representation (VOIR)) to create 3D VOIR data.

6. The system of claim 5, further comprising a using self-supervised TransVW model with ((D)+R)+A) on the 3D VOIR data to improve PE false positive reduction performance for subsequent slice-level PE classification.

7. The system method of claim 1, wherein performing and outputting an exam-level diagnosis using E-ViT comprises generating predictions for a collection of the slices and assigning a label to each corresponding exam.

8. The system of claim 1, further comprising using a Models Genesis self-supervised learning method to improve PE false positive reduction performance for subsequent slice-level PE classification.

9. A non-transitory computer readable storage media having instructions stored thereupon that, when executed by a system having at least a processor and a memory therein, cause the processor to perform the following operations:
receiving a plurality of Computed Tomography Pulmonary Angiography (CTPA) exams (hereinafter the “exams”) as input image data, each exam in the plurality of exams comprising a varying plurality of individual images (hereinafter the “slices”);
annotating each of the slices with one of a pulmonary embolism (PE) present label or PE absent label;
annotating each of the exams with one of a plurality of labels each indicating a different PE state and location;
performing a slice-level PE classification to determine the presence or absence of PE for each of the slices; and
performing and outputting, via an Embedding-based Vision Transformer (E-ViT), an exam-level diagnosis using the slice-level classifications.

10. The non-transitory computer readable storage media of claim 9, wherein the plurality of exams comprises one of the Radiological Society of North America (RSNA) Pulmonary Embolism (PE) CT dataset and the Computer Aided Diagnosis-Pulmonary Embolism (CAD-PE) Challenge dataset, and an in-house PE-CAD dataset.

11. The system of claim 9, wherein performing slice-level PE classification to determine the presence or absence of PE for each of the slices, comprises pre-processing steps of lung localization to focus on a region of interest in the slices, and windowing to highlight pixel intensities within a range of 100-700 Hounsfield Units.

12. The system of claim 9, wherein the plurality of exams comprises the RSNA PE CT dataset, and wherein performing slice-level PE classification to determine the presence or absence of PE for each of the slices comprises performing slice-level classification using an ensemble of CNN-based architectures including Xception, SeXception and SeResNext50.

13. The system of claim 9, wherein the plurality of exams comprises the CAD-PE Challenge dataset, and wherein performing slice-level PE classification to determine the presence or absence of PE for each of the slices comprises a pre-processing step of representing the input image data using a three-dimensional (3D) vessel-oriented image representation (VOIR)) to create 3D VOIR data.

14. The system of claim 13, further comprising a using self-supervised TransVW model with ((D)+R)+A) on the 3D VOIR data to improve PE false positive reduction performance for subsequent slice-level PE classification.

15. The system method of claim 9, wherein performing and outputting an exam-level diagnosis using E-ViT comprises generating predictions for a collection of the slices and assigning a label to each corresponding exam.

16. The system of claim 9, further comprising using a Models Genesis self-supervised learning method to improve PE false positive reduction performance for subsequent slice-level PE classification.

17. A computer-implemented method, comprising:
receiving a plurality of Computed Tomography Pulmonary Angiography (CTPA) exams (hereinafter the “exams”) as input image data, each exam in the plurality of exams comprising a varying plurality of individual images (hereinafter the “slices”);
annotating each of the slices with one of a pulmonary embolism (PE) present label or PE absent label;
annotating each of the exams with one of a plurality of labels each indicating a different PE state and location;
performing a slice-level PE classification to determine the presence or absence of PE for each of the slices; and
performing and outputting, via an Embedding-based Vision Transformer (E-ViT), an exam-level diagnosis using the slice-level classifications.

18. The computer-implemented method of claim 17 wherein the plurality of exams comprises one of the Radiological Society of North America (RSNA) Pulmonary Embolism (PE) CT dataset and the Computer Aided Diagnosis-Pulmonary Embolism (CAD-PE) Challenge dataset, and an in-house PE-CAD dataset.

19. The computer-implemented method of claim 18, wherein performing slice-level PE classification to determine the presence or absence of PE for each of the slices, comprises pre-processing steps of lung localization to focus on a region of interest in the slices, and windowing to highlight pixel intensities within a range of 100-700 Hounsfield Units.

20. The computer-implemented method of claim 18, wherein the plurality of exams comprises the RSNA PE CT dataset, and wherein performing slice-level PE classification to determine the presence or absence of PE for each of the slices comprises performing slice-level classification using an ensemble of CNN-based architectures including Xception, SeXception and SeResNext50.
